(19) United States
(12) Patent Application Publication
(10) Pub. No.: US 2019/0059739 A1
(43) Pub. Date: Feb. 28, 2019

(54) PHOTOACOUSTIC APPARATUS

(71) Applicant: CANON KABUSHIKI KAISHA, Tokyo (JP)

(72) Inventor: Naoto Abe, Machida-shi (JP)

(21) Appl. No.: 16/112,382

(22) Filed: Aug. 24, 2018

(30) Foreign Application Priority Data
Aug. 31, 2017 (JP) 2017-167255
Aug. 31, 2017 (JP) 2017-167256

(51) Int. Cl. A61B 5/00; A61B 5/145

(52) U.S. Cl. CPC A61B 5/0095; A61B 5/14542

(57) ABSTRACT

A photoacoustic apparatus is provided that is capable of widening the frequency bandwidth of detected acoustic waves. The photoacoustic apparatus includes a control unit for controlling a plurality of light irradiation units to irradiate pulsed lights at least a part of which have pulse waveforms different from pulse waveforms of the other pulsed lights onto the subject a plurality of times or controlling the plurality of the light irradiation units so that the pulsed lights to be emitted from the plurality of light irradiation units have pulse waveforms different from each other.

### BACKGROUND OF THE INVENTION

### Field of the Invention

The present invention relates to a photoacoustic apparatus.

### Description of the Related Art

In recent years, photoacoustic apparatuses for imaging the inside of a subject by using a photoacoustic effect have been studied and developed as a light-based imaging technique. A photoacoustic apparatus acquires information about a subject based on ultrasonic waves (photoacoustic waves) generated through a photoacoustic effect from an optical absorber which has absorbed the energy of light radiated onto the subject.

Japanese Patent Application Laid-Open No. 2017-46823 discusses a photoacoustic imaging apparatus for setting the ratio of the inclination of the rising edge to the inclination of the falling edge of an optical pulse waveform emitted from a light source unit for conformance with the frequency bandwidth of a detection unit for detecting an acoustic wave.

The technique discussed in Japanese Patent Application Laid-Open No. 2017-46823 uses only the inclination ratio as a parameter for changing the detection frequency, and therefore the increase in the detection frequency bandwidth has been limited.

### SUMMARY OF THE INVENTION

According to an aspect of the present invention, a photoacoustic apparatus includes a light irradiation unit configured to irradiate a subject with pulsed light, a reception unit configured to receive an acoustic wave generated when the subject is irradiated with the pulsed light and to convert the acoustic, wave into an electrical signal, a control unit configured to control the light irradiation unit to irradiate the subject with a plurality of optical pulses of the pulsed light, and an acquisition unit configured to acquire information about the subject based on a plurality of the electrical signals converted from a plurality of acoustic waves generated when the subject is irradiated with the pulsed light a plurality of times, wherein the control unit controls the light irradiation unit so that at least a part of the optical pulses to be emitted a plurality of times have a pulse waveform different from a pulse waveform of the other optical pulses.

According to another aspect of the present invention, a photoacoustic apparatus includes a plurality of light irradiation units configured to irradiate a subject with pulsed light, a control unit configured to control the plurality of light irradiation units to irradiate the subject with the pulsed light from the plurality of light irradiation units at approximately the same time, a reception unit configured to receive an acoustic wave generated when the subject is irradiated with the pulsed light and convert the acoustic wave into an electrical signal, and an acquisition unit configured to acquire information about the subject based on the electrical signal, wherein the control unit controls the plurality of light irradiation units to provide the optical pulses emitted from the plurality of light irradiation units with pulse waveforms different from each other.

Further features of the present invention will become apparent from the following description of exemplary embodiments with reference to the attached drawings.

### BRIEF DESCRIPTION OF THE DRAWINGS

FIGS. 1A to 1H are graphs each illustrating an action and an effect of a photoacoustic apparatus according to an exemplary embodiment of the present invention.

FIG. 2 is a block diagram illustrating the photoacoustic apparatus according to the exemplary embodiment of the present invention.

FIG. 3 is a diagram illustrating a structure a probe according to the exemplary embodiment of the present invention.

FIGS. 4A and 4B are diagrams each illustrating an example of a configuration of a drive unit according to the exemplary embodiment of the present invention.

FIG. 5 is a block diagram illustrating an example of a configuration of a computer according to the exemplary embodiment of the present invention.

FIG. 6 is a diagram illustrating an example of control of the photoacoustic apparatus according to the exemplary embodiment of the present invention.

FIGS. 7A to 7J are diagrams each illustrating an action and an effect of a photoacoustic apparatus according to another exemplary embodiment of the present invention.

FIG. 8 is a block diagram illustrating the photoacoustic apparatus according to an exemplary embodiment of the present invention.

FIG. 9 is a diagram illustrating a structure of a probe according to an exemplary embodiment of the present invention.

FIGS. 10A and 10B are diagrams illustrating an example of a configuration of a drive unit according to an exemplary embodiment of the present invention.

FIG. 11 is a block diagram illustrating another example of a configuration of a computer according to an exemplary embodiment of the present invention.

FIG. 12 is a diagram illustrating another example of control of the photoacoustic apparatus according to an exemplary embodiment of the present invention.

### DESCRIPTION OF THE EMBODIMENTS

An exemplary embodiment of the present invention will be described below with reference to the accompanying drawings. However, sizes, materials, shapes, and relative arrangements of elements described below are not limited thereto and can be modified depending on the configuration of an apparatus according to the present exemplary embodiment and other various conditions. Therefore, the scope of the present exemplary embodiment is not limited to the following descriptions.

The present exemplary embodiment relates to a technique for detecting acoustic waves transmitted from a subject and generating and acquiring characteristic information about the inside of the subject. Therefore, the present exemplary embodiment can also be regarded as a subject information acquisition apparatus and a method for controlling the subject information acquisition apparatus or regarded as a subject information acquisition method and a signal processing method. The present exemplary embodiment can also be regarded as a method for generating and displaying an image which indicates intra-subject characteristic information. The present exemplary embodiment can also be regarded as a program for causing an information processing apparatus having hardware resources such as a central processing unit (CPU) and a memory to execute these methods or regarded as a computer-readable non-transitory storage medium storing the program,

As an example of a subject information acquisition apparatus according to the present exemplary embodiment, a photoacoustic imaging apparatus irradiates a subject with light (electromagnetic wave) a plurality of times, receives acoustic waves generated within the subject in respective irradiations, and acquires characteristic information about the inside of the subject as image data based on the received photoacoustic signals, by using a photoacoustic effect. Another example of the subject information acquisition apparatus according to the present exemplary embodiment irradiates a subject with optical pulses (electromagnetic waves) by using a plurality of light irradiation units and acquires characteristic information about the inside of the subject as image data based on the received photoacoustic signals, by using a photoacoustic effect. The subject information acquisition apparatus according to the present exemplary embodiment is a photoacoustic apparatus for simultaneously emitting optical pulses having at least two different optical pulse waveforms in a plurality of light emissions from a light irradiation unit and acquiring information based on desired frequency characteristics of photoacoustic signals.

In each of the above-described examples, the characteristic information refers to information of characteristic values, each corresponding to a different one of a plurality of positions inside the subject, generated by using signals resulting from received photoacoustic waves.

Photoacoustic image data according to the present exemplary embodiment is a concept including all types of image data resulting from photoacoustic waves generated through light irradiation. Examples of photoacoustic image data include image data representing the spatial distribution of at least one piece of subject information such as the generated sound pressure (initial sound pressure), absorbed energy density, and absorption coefficient of a photoacoustic wave, and the density (oxygen saturation) of the constituent of a subject. Photoacoustic image data indicating spectral information such as the density of the constituent of a subject based on signals (photoacoustic signals) resulting from photoacoustic waves generated in a plurality of light emissions which are mutually different in wavelength. Photoacoustic image data indicating spectral information may include oxygen saturation, oxygen saturation weighted by the strength such as the absorption coefficient, total hemoglobin density, oxyhemoglobin density, or deoxyhemoglobin density. Photoacoustic image data indicating spectral information may be the volume fraction of glucose density, collagen density, melanin density, fat, or water.

A two- or three-dimensional characteristic information distribution can be obtained based on characteristic information at each position inside the subject. Distribution data may be generated as image data. Characteristic information may be obtained not as numerical data but as distribution information at each position inside the subject. More specifically, the characteristic information is distribution information such as the initial sound pressure distribution, energy absorption density distribution, absorption coefficient distribution, and oxygen saturation distribution.

An acoustic wave according to the present exemplary embodiment typically an ultrasonic wave and includes an elastic wave which is called a sound wave or an acoustic wave. An electrical signal converted from an acoustic wave by a transducer or the like is also referred to as an acoustic signal. However, in the present specification, a description of an ultrasonic wave or an acoustic wave is not intended to limit the wavelength of the elastic waves. An acoustic wave generated by a photoacoustic effect is referred to as a photoacoustic wave or an optical ultrasonic wave. An electrical signal resulting from a photoacoustic wave is also referred to as a photoacoustic signal. Distribution data is also referred to as photoacoustic image data or reconstructed image data.

A first exemplary embodiment will be described below centering on a photoacoustic apparatus, as an example of a subject information acquisition apparatus, for irradiating a subject with pulsed light a plurality of times, receiving photoacoustic waves from the subject, and acquiring information about inside of the subject, such as blood vessel image (structure image). Although the present exemplary embodiment focuses on a photoacoustic apparatus having a handheld type probe, the present exemplary embodiment is also applicable to a photoacoustic apparatus having a probe on a stage for mechanical scanning. The present exemplary embodiment will be described below centering on a photoacoustic apparatus having a handheld type probe that includes a plurality of semiconductor light emission elements and that is wired to the apparatus main body. However, the present exemplary embodiment is also applicable to a photoacoustic apparatus having a handheld type probe, having a power source such as a battery, for wirelessly transmitting photoacoustic signals of the handheld type probe to the photoacoustic apparatus main body. If the handheld type probe has the functions of the present exemplary embodiment as described above, the photoacoustic apparatus according to the present exemplary embodiment means the handheld type probe itself.

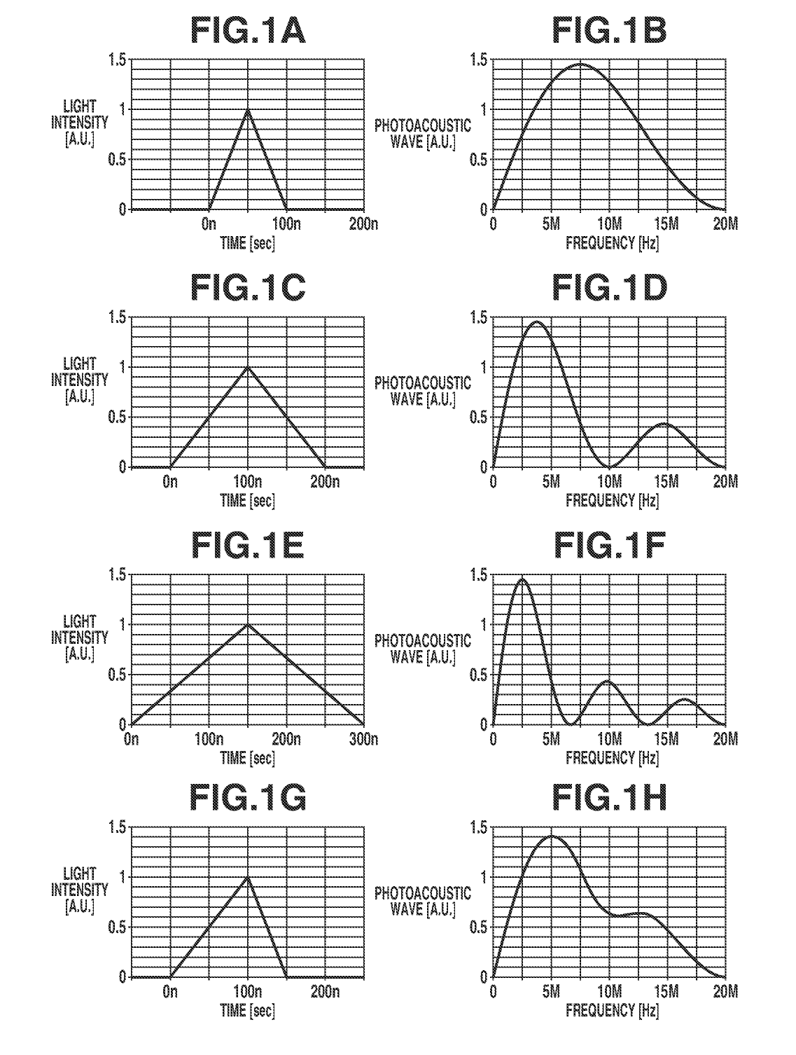

The basic concept of the present exemplary embodiment will be described below. Reference numerals used in the descriptions of the first exemplary embodiment correspond to those illustrated in FIGS. 1A to 6.

FIGS. 1A to 1H are graphs illustrating relations between optical pulse waveforms and photoacoustic waves (photoacoustic signals) for describing the basic concept of the present exemplary embodiment. FIGS. 1A and 1C are graphs illustrating optical pulse waveforms, where the vertical axis indicates the light intensity and the horizontal axis indicates time. FIGS. 1B and 1D are graphs illustrating simulations of the frequency characteristics (frequency spectrum) of photoacoustic waves obtained when the subject 100 is irradiated with the optical pulse waveforms illustrated in FIGS. 1A and 1C, respectively. The vertical axis indicates the photoacoustic wave intensity and the horizontal axis indicates the frequency. When the subject 100 is irradiated with an optical pulse having a short pulse width (100 ns), as illustrated in FIG. 1A, the frequency spectrum peak value of the obtained photoacoustic wave is about 7.5 MHz, as illustrated in FIG. 1B. On the other hand, when the subject 100 is irradiated with an optical pulse having a long pulse width (200 ns), as illustrated in FIG. 1C, the frequency spectrum peak value of the obtained photoacoustic wave is about 3.75 MHz, as illustrated in FIG. 1D.

According to the first exemplary embodiment, the photoacoustic apparatus 1 irradiates the subject 100 with optical pulses having different optical pulse waveforms a plurality of times and acquires information about photoacoustic signals based on photoacoustic signals obtained in the light irradiations. For example, the photoacoustic apparatus 1 emits light 12 times, more specifically, the photoacoustic apparatus 1 irradiates the subject 100 six times with an optical pulse having the optical pulse waveform illustrated in FIG. 1A and then irradiates the subject 100 another six times with an optical pulse having the optical pulse waveform illustrated in FIG. 1C. Then, the photoacoustic apparatus 1 performs addition averaging of the photoacoustic signals acquired through the light irradiations. The result makes it possible to obtain a photoacoustic signal having the frequency spectrum of a photoacoustic wave as a result of addition-averaging of the frequency spectra of the photoacoustic waves illustrated in FIGS. 1B and 1D.

As another method, controlling the number of light emissions enables easily changing the frequency spectrum of a photoacoustic wave. For example, the photoacoustic apparatus 1 emits light 12 times, more specifically, it irradiates the subject 100 four times with an optical pulse having the optical pulse waveform illustrated in FIG. 1A and then irradiates the subject 100 eight times with an optical pulse having the optical pulse waveform illustrated in FIG. 1C. Then, the photoacoustic apparatus 1 performs addition averaging of the photoacoustic signals acquired through the light irradiations. The result makes it possible to obtain a combined photoacoustic signal representing the frequency spectrum of a photoacoustic wave as a result of performing addition-averaging of the frequency spectra of the photoacoustic waves illustrated in FIGS. 1B and 1D, which are weighted with a ratio of 1:2, respectively. In this case, the subject 100 is irradiated with an optical pulse having the optical pulse waveform illustrated in FIG. 1C a large number of times. Therefore, high-frequency components of the frequency characteristics of the combined photoacoustic signal can be relatively increased in comparison with the case where the number of light emissions remains unchanged for the two different optical pulse waveforms.

As another method, controlling the light intensity also enables easily changing the frequency spectrum of a photoacoustic wave. For example, the photoacoustic apparatus 1 emits light 12 times, more specifically, the photoacoustic apparatus 1 irradiates the subject 100 six times with an optical pulse having the optical pulse waveform illustrated in FIG. 1A and then irradiates the subject 100 another six times with an optical pulse having a light intensity twice that of the optical pulse waveform illustrated in FIG. 1C. Then, the photoacoustic apparatus 1 performs addition averaging of the photoacoustic signals acquired through the light irradiations. The result makes it possible to obtain a combined photoacoustic signal representing the frequency spectrum of a photoacoustic wave as a result of averaging the frequency spectra of the photoacoustic waves illustrated in FIGS. 1B and 1D, which are weighted with a ratio of 1:2, respectively. In this case, the subject 100 is irradiated with an optical pulse having the optical pulse waveform illustrated in FIG. 1C with a high light intensity. Therefore, high-frequency components of the frequency characteristics of the combined photoacoustic signal can be increased in comparison with the case where the light intensity remains unchanged for the two different optical pulse waveforms. As another method for controlling the light intensity in a case where a light irradiation unit includes a light source unit including a plurality of semiconductor light emission elements, the light intensity may be controlled by increasing or decreasing the number of semiconductor light emission elements for emitting pulsed light.

As another method, controlling the gain of an amplifier for amplifying a photoacoustic signal received in each light emission enables easily changing the frequency spectrum of a photoacoustic wave. For example, the photoacoustic apparatus 1 emits light 12 times, more specifically, the photoacoustic apparatus 1 irradiates the subject 100 six times with an optical pulse having the optical pulse waveform illustrated in FIG. 1A and then irradiates the subject 100 another six times with an optical pulse having the optical pulse waveform illustrated in FIG. 1C. In this case, the photoacoustic apparatus 1 amplifies with a double gain the photoacoustic signal obtained by irradiating the subject 100 with an optical pulse having the optical pulse waveform illustrated in FIG. 1A and amplifies with a ×1 gain the photoacoustic signal obtained by irradiating the subject 100 with an optical pulse having the optical pulse waveform illustrated in FIG. 1C. Such a gain may be implemented by changing the gain of an analog amplifier or by digitizing the photoacoustic signal and then performing digital operations through a CPU. Then, photoacoustic signals acquired and amplified for each irradiation are addition-averaged. The result makes it possible to obtain a combined photoacoustic signal representing the frequency spectrum of a photoacoustic wave as a result of averaging the frequency spectra of the photoacoustic waves illustrated in FIGS. 1B and 1D, which are weighted with a ratio of 2:1, respectively. In this case, the photoacoustic signal obtained by irradiating the subject 100 with an optical pulse having the optical pulse waveform illustrated in FIG. 1A increases. Therefore, low-frequency components of the frequency characteristics of the combined photoacoustic signal can be relatively increased in comparison with the case where the gain remains unchanged for the two different optical pulse waveforms,

As another method, three or more optical pulse waveforms can be used. For example, the waveform of a third optical pulse is illustrated in FIG. 1L. FIG. 1F is a graph illustrating a simulation of the frequency spectrum of a photoacoustic wave obtained by irradiating the subject 100 with the optical pulse waveform illustrated in FIG. 1E. Referring to the graph illustrated in FIG. 1E, the vertical axis indicates the light intensity and the horizontal axis indicates time. Referring to the graph illustrated in FIG. 1F, the vertical axis indicates the intensity of the photoacoustic wave and the horizontal axis indicates the frequency. When the subject 100 is irradiated with an optical pulse having a 300 ns pulse width, as illustrated in FIG. 1E, the frequency spectrum peak value of the obtained photoacoustic wave is about 2.5 MHz, as illustrated in FIG. 1F. For example, the photoacoustic apparatus 1 emits light 12 times, more specifically, the photoacoustic apparatus 1 irradiates the subject 100 four times with an optical pulse having the optical pulse waveform illustrated in FIG. 1A, irradiates the subject 100 another four times with an optical pulse having the optical pulse waveform illustrated in FIG. 1C, and then irradiates the subject 100 yet another four times with an optical pulse having the optical pulse waveform illustrated in FIG. 1E. Then, the photoacoustic apparatus 1 performs addition averaging of the photoacoustic signals acquired through the light irradiations. The result makes it possible to obtain a combined photoacoustic signal representing the frequency spectrum of a photoacoustic wave as a result of averaging the frequency spectra of the photoacoustic waves illustrated in FIGS. 1B, 1D, and 1F. As a result of irradiating the subject 100 with three or more different optical pulse waveforms, as described above, it becomes possible to raise high-frequency components of the frequency characteristics of the combined photoacoustic signal.

Similar to the above descriptions, controlling the number of light emissions and the light intensity of three or more different optical pulse waveforms or controlling the gain for amplifying a photoacoustic signal received in each light emission enables more finely controlling the frequency characteristics of the combined photoacoustic signal.

According to the present exemplary embodiment, as described above, the shapes of the optical pulse waveforms are set in such a manner that frequencies of frequency spectrum peak values of photoacoustic waves to be obtained by using optical pulses having at least two different optical pulse waveforms are different from each other. Such a configuration suitably makes it easier to adjust the frequency characteristics of a photoacoustic signal. To provide different frequencies of frequency spectrum peak values of photoacoustic waves, it is desirable to set pulse widths mutually different from each other when optical pulse waveforms have a triangle form in. More specifically, it is desirable to select the pulse width of optical pulse waveforms from 100 to 1000 ns. For example, triangle waves having a 100 ns pulse width and a. 200 ns pulse width may be used, or triangle waves having a 100 ns pulse width and a 300 ns pulse width may be used. In addition, the above-described three different triangle waves may be used.

An optical pulse having an asymmetrical optical pulse waveform with a rising time of 100 ns and a falling time of 50 ns, as illustrated in FIG. 1G, may be used. In this case, a frequency spectrum of a photoacoustic wave as illustrated in FIG. 1H results. If such characteristics are taken into consideration, naturally, this frequency spectrum is naturally applicable to the present exemplary embodiment. Optical pulse waveforms having any shapes are applicable to the present exemplary embodiment.

Control of the frequency characteristics of a combined photoacoustic signal has been described to be possible. According to the present exemplary embodiment, as discussed in Japanese Patent Application Laid-Open No. 2017-46823, it is also possible to control the number of light emissions, light intensity, gain, etc. to adjust the frequency characteristics of a combined photoacoustic signal to the frequency bandwidth of a reception unit. In the case of a photoacoustic apparatus with which a probe is exchangeable, it is desirable to control the number of light emissions, light intensity, gain, etc. to adjust the frequency characteristics of the combined photoacoustic signal to the frequency characteristics of the probe. The number of light emissions, light intensity, gain, etc. may be determined so that an intensity of the frequency spectrum of a photoacoustic wave which is determined depending on the thickness of a blood vessel in a subject region under observation becomes high. The number of light emissions, light intensity, gain, etc. may be controlled to enable controlling the frequency characteristics of a photoacoustic signal according to a user instruction. For example, when a user wants to observe a thin blood vessel, the following setting may be made: the number of light emissions of an optical pulse having a short optical pulse width is increased to increase high-frequency components of the frequency characteristics of a photoacoustic signal. For example, when the user wants to observe a thick blood vessel, the following setting may be made: the number of light emissions of an optical pulse having a long optical pulse width is increased to increase low-frequency components of the frequency characteristics of a photoacoustic signal. When the subject region under observation is deep inside the subject, high-frequency components of photoacoustic waves are largely attenuated. In this case, therefore, a computer may automatically set the shape of an optical pulse waveform and the number of light emissions to increase high-frequency components of the frequency characteristics of a photoacoustic signal. It is also possible to provide a pressure sensor on a handheld type probe and measure the pressing force with which the probe is pressed onto the subject 100. In this case, the computer may change the frequency characteristics of a photoacoustic signal depending on the magnitude of the pressing force. For example, when observing details, the user may strongly press the probe onto the subject in an unconscious way. In the case of a large pressing force, the number of light emissions of an optical pulse having a short optical pulse width, the light intensity, or the gain may be increased to increase high-frequency components of the frequency spectrum of a photoacoustic wave.

In the above-described example, the photoacoustic apparatus 1 irradiates the subject 100 with optical pulses having different optical pulse waveforms a plurality of times, and addition-averages obtained photoacoustic signals in the light irradiations to obtain a combined photoacoustic signal having characteristics of averaged frequency spectra of the photoacoustic waves. As a method for combining photoacoustic signals applicable to the present exemplary embodiment, the following method is also useful. For example, it is also possible to weight the acquired photoacoustic signals in proportion to the light intensity of the frequency spectrum of each photoacoustic wave obtained based on the radiated optical pulse waveforms and then perform addition averaging of acquired photoacoustic signals. This combination method obtains an average with which components with a high light intensity and accordingly a high signal-to-noise (S/N) ratio are weighted to a further extent, providing a favorable S/N ratio. When combining photoacoustic signals, it is also possible to select portions having a high light intensity of the frequency spectrum of each photoacoustic wave according to the optical pulse waveform. More specifically, bandwidths of photoacoustic signals subjected to the combination may be determined based on the frequency spectra of the photoacoustic waves according to at least two different optical pulse waveforms. The following assumes an example case where the optical pulse waveforms illustrated in FIGS. 1A and 1C, are used. For frequencies of 5 MHz or lower, it is desirable to use a signal as a result of addition-averaging of photoacoustic signals acquired by irradiating the subject 100 with the optical pulse waveform illustrated in FIG. 1A. For frequencies of 5 MHz or higher, it is desirable to use a signal as a result of addition-averaging of photoacoustic signals acquired by irradiating the subject 100 with the optical pulse waveform illustrated in FIG. 1C. Naturally, to avoid unnatural transitions between changing frequencies, it is desirable to perform combination processing with weighted photoacoustic signals rather than simple frequency changeover.

It is also possible to, before performing addition averaging, flatten the frequency spectra of the photoacoustic waves obtained in emissions of light having at least two different optical pulse waveforms by using a filter having characteristics for providing a flat frequency spectrum of a photoacoustic wave according to an optical pulse waveform. Although this method does not improve the S/N ratio, it has an advantage that the combined photoacoustic signal has fiat frequency characteristics.

As described above, in a photoacoustic apparatus for acquiring information based on photoacoustic signals generated by a plurality of pulsed light emissions from the light irradiation unit, light having at least two different optical pulse waveforms is emitted in a plurality of light emissions from the light irradiation unit. Driving the semiconductor light emission elements included in the light irradiation unit to emit light in this way enables acquiring information based on the predetermined (desired) frequency characteristics of a photoacoustic signal. The following describes an exemplary embodiment in which photoacoustic signals acquired through two different optical pulse waveforms are addition-averaged. Naturally, the exemplary embodiment is also applicable to a configuration in which the above-described processing is performed on photoacoustic signals acquired by irradiating the subject 100 with three or more different pulse waveforms.

### (Configuration of Photoacoustic Apparatus)

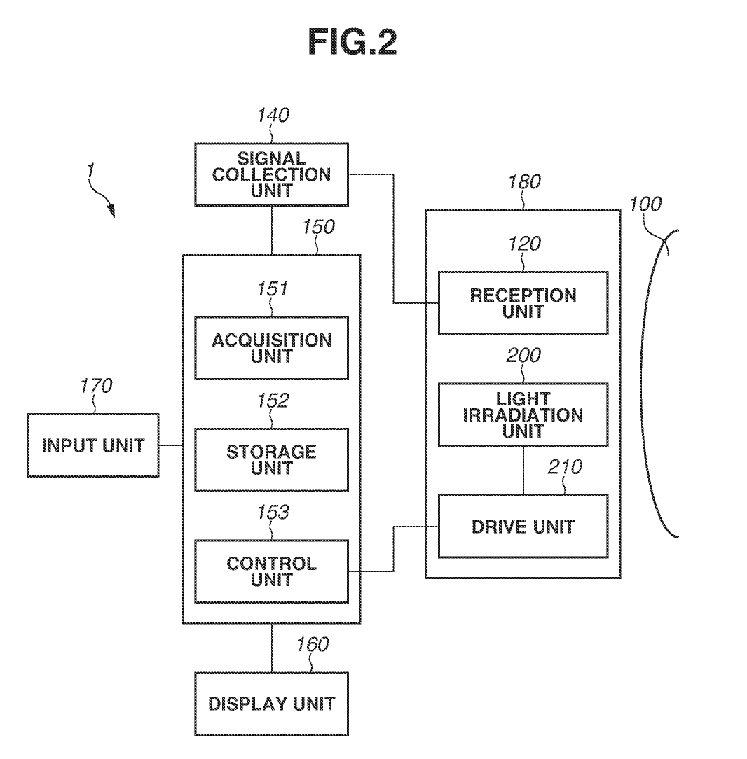

FIG. 2 is a block diagram illustrating a photoacoustic apparatus 1 according to the present exemplary embodiment. Referring to the block diagram illustrated in FIG. 2, the photoacoustic apparatus 1 according to the present exemplary embodiment includes a light irradiation unit 200 for irradiating the subject 100 with pulsed light generated by a light source, and a reception unit 120 for receiving acoustic waves generated when the subject 100 is irradiated with the pulsed light and converting the acoustic waves into electrical signals. The photoacoustic apparatus 1 further includes a control unit 153 for controlling the light irradiation unit 200 to irradiate the subject 100 with pulsed light a plurality of times. The photoacoustic apparatus 1 further includes an acquisition unit 151 for acquiring information about the subject 100 based on a plurality of the electrical signals converted from the acoustic waves generated when the subject 100 is irradiated with pulsed light a plurality of times. The control unit 153 controls the light irradiation unit 200 so that at least a part of the optical pulses to be emitted a plurality of times has different pulse waveforms. As described above, the frequency of each of the detected acoustic waves changes because the optical pulses radiated onto the subject 100 have different pulse waveforms. Therefore, the frequency bandwidth of the detected acoustic waves can be widened by increasing the number of types of pulse waveforms of the optical pulses to be radiated onto the subject 100. This makes it possible to acquire information about substances ranging from a large-sized substance corresponding to a low-frequency acoustic wave to a small-sized substance corresponding to a high-frequency acoustic wave out of optical absorbers distributed over the subject 100.

Although, in the present exemplary embodiment, different pulse waveforms typically correspond to different pulse widths, different pulse waveforms are not limited thereto.

According to the present exemplary embodiment, the control unit 153 is able to control the light irradiation units to emit optical pulses having different pulse waveforms different number of times and to control the light irradiation units to emit optical pulses having different pulse waveforms in different light intensities. When generating a large acoustic wave having a specific frequency in the above-described configuration, it is possible to increase the number of light emissions of pulsed light having the pulse waveform for generating the specific frequency and increase the light intensity of the pulsed light.

The photoacoustic apparatus 1 according to the present exemplary embodiment includes a probe 180, a signal collection unit 140, a computer 150, a display unit 160, and an input unit 170. The probe 180 includes a light irradiation unit 200, a drive unit 210, and a reception unit 120. The computer 150 includes an acquisition unit 151, a storage unit 152, and a control unit 153. Portions connected with cables, other than the probe 180, are also collectively referred to as a photoacoustic apparatus main body.

According to the present exemplary embodiment, the light irradiation unit 200 includes a light source unit for generating pulsed light. The light source unit includes a plurality of semiconductor light emission elements. It is desirable that a plurality of semiconductor fight emission elements is used to raise the light intensity to improve the S/N ratio of photoacoustic signals. However, one semiconductor light emission element may be used to achieve the effect of the present exemplary embodiment. The drive unit 210 drives a plurality of the semiconductor light emission elements of the light irradiation unit 200 to emit light having the above-described optical pulse waveforms. The drive unit 210 controls the optical pulse waveform from the light irradiation unit 200 in each light emission. It is desirable to determine the optical pulse waveform as described above.

The drive unit 210 controls the semiconductor light emission elements of the light irradiation unit 200 to emit light having at least two different optical pulse waveforms a plurality of times. Then, the light irradiation unit 200 irradiates the subject 100 with light having at least two different optical pulse waveforms. The reception unit 120 receives a photoacoustic wave generated within the subject 100 in each light emission and outputs an analog electrical signal (photoacoustic signal). The signal collection unit 140 converts the analog signal output from the reception unit 120 in each light emission into a digital signal and outputs the digital signal to the computer 150.

The computer 150 combines the digital signals output from the signal collection unit 140 in respective light emissions by using the acquisition unit 151, the storage unit 152, and the control unit 153 and stores the resultant signal in the storage unit 152 as an electrical signal (combined photoacoustic signal) resulting from photoacoustic waves. Combining signals includes not only simple addition averaging but also the above-described processing. Although the following descriptions will be made centering mainly on addition averaging, the above-described combination method other than addition averaging is also applicable. The computer 150 performs such processing as image reconstruction on the digital signal stored in the storage unit 152 to generate photoacoustic image data. Then, the photoacoustic image data is displayed on the display unit 160. The computer 150 also controls operations of the entire photoacoustic apparatus 1 and performs optical pulse waveform setting, light emission control, and reception control. The computer 150 may perform image processing for display on the obtained photoacoustic image data and processing for combining graphics data for graphical user interfaces (GUIs, not illustrated).

The user (doctor or engineer) can perform diagnosis by checking a photoacoustic image displayed on the display unit 160. Based on a storage instruction front the user or the computer 150, the display image may be stored in a memory of the computer 150 or in a data management system connected with the photoacoustic apparatus 1 via a network. The input unit 170 receives an instruction from the user.

### (Detailed Configuration of Each Block)

A desirable configuration of each block w be described in detail below.

### (Probe 180)

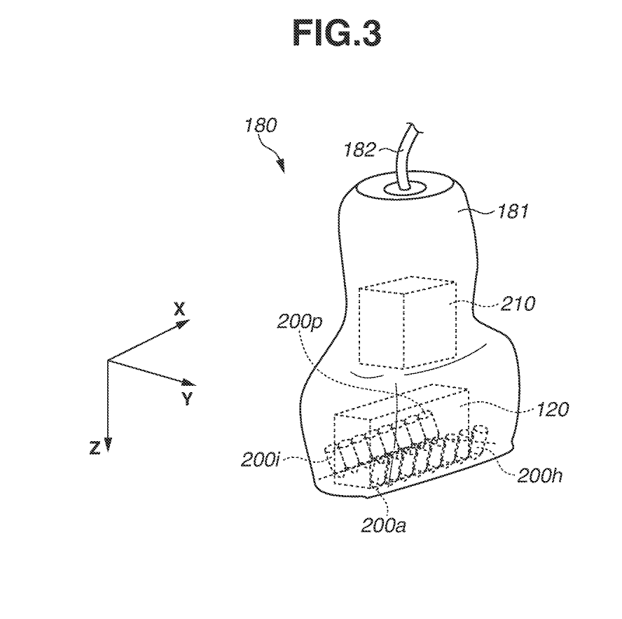

FIG. 3 illustrates a structure of the probe 180 according to the first exemplary embodiment.

Referring to FIG. 3, the probe 180 includes a light irradiation unit 200, a drive unit 210, a reception unit 120, and a housing 181. The housing 181 surrounds the light irradiation unit 200, the drive unit 210, and the reception unit 120. Grasping the housing 181, the user can use the probe 180 as a handheld type probe.

The light irradiation unit 200 irradiates the subject 100 with optical pulses. As described above, the drive unit 210 is controlled by the light irradiation unit 200 to emit an optical pulse having a desired optical pulse waveform in each optical pulse emission.

The X, Y, and Z axes illustrated in FIG. 3 indicate coordinate axes when the probe 180 is statically placed and do not limit the direction of when the probe 180 is being used.

The probe 180 illustrated in FIG. 3 is connected with the signal collection unit 140 via a cable 182. The cable 182 includes a wiring for supplying power to the light irradiation unit 200, a wiring for a light emission control signal, and a wiring for outputting the analog signal from the reception unit 120 to the signal collection unit 140. A connector may be provided on the cable 182 to separate the probe 180 from other components of the photoacoustic apparatus 1.

### (Light Irradiation Unit 200)

The light irradiation unit 200 according to the present exemplary embodiment irradiates the subject 100 with pulsed light generated by the light source.

The light irradiation unit 200 includes, for example, 16 semiconductor light emission elements such as laser diodes (LDs). The semiconductor light emission elements are not limited to LDs and may be light emitting diodes (LEDs). The type and the number of semiconductor light emission elements are determined by the specified light intensity. Eight laser diodes (200a to 200h) and another eight laser diodes (200i to 200p) are disposed to face each other across the reception unit 120. These laser diodes irradiate the subject 100 with light. Each laser diode is mounted toward the maximum sensitivity direction of the reception unit 120. More specifically, as illustrated in FIG. 3, the laser diodes 200a to 200h and the laser diodes 200i to 200p are mounted in such a manner that the laser diodes 200a to 200h and the laser diodes 200i to 200p incline toward the direction of the reception unit 120. Although, in FIG. 3, the reception unit 120 is mounted between two sets of the laser diodes, light sources may be collectively disposed on one side. In addition, more than 16 laser diodes may be used. Although, in FIG. 3, discrete parts are arranged, a plurality of dies cut out from a semiconductor wafer may be mounted on a metal base or printed circuit board. The configuration of the light irradiation unit 200, i.e., the arrangement of the laser diodes is not limited to the above-described arrangement. The laser diodes may be mounted in any arrangement depending on the probe shape or other conditions as long as the subject 100 can be favorably irradiated with optical pulses.

For example, it is desirable that the pulse width of light emitted from the light irradiation unit 200 is 10 ns or more and 1 μs or less, more desirably, 100 ns or more and 800 ns or less. Although the wavelength of the light of the light irradiation unit 200 is desirably 400 nm or more and 1600 nm or less, it is desirable to determine the wavelength according to the optical absorption characteristics of the optical absorber subjected to imaging. When imaging a blood vessel with a high resolution, light having a wavelength (400 or more and 800 nm or less) which is largely absorbed in a blood vessel may be used. When imaging a deep inside of a living body, light having a wavelength (700 nm or more and 1100 nm or less) which is absorbed little in a background tissue (water, fat, etc.) of a living body may be used. According to the present exemplary embodiment, a predetermined wavelength is set to 975 nm which allows acquisition of blood vessel structure information and is reachable to deep portions of the subject 100. A photoacoustic wave equivalent to that acquired through the emission of the predetermined wavelength can be acquired by simultaneously irradiating the subject 100 with light of a plurality of light sources having other wavelengths satisfying the above-described condition.

### (Driving Unit 210)

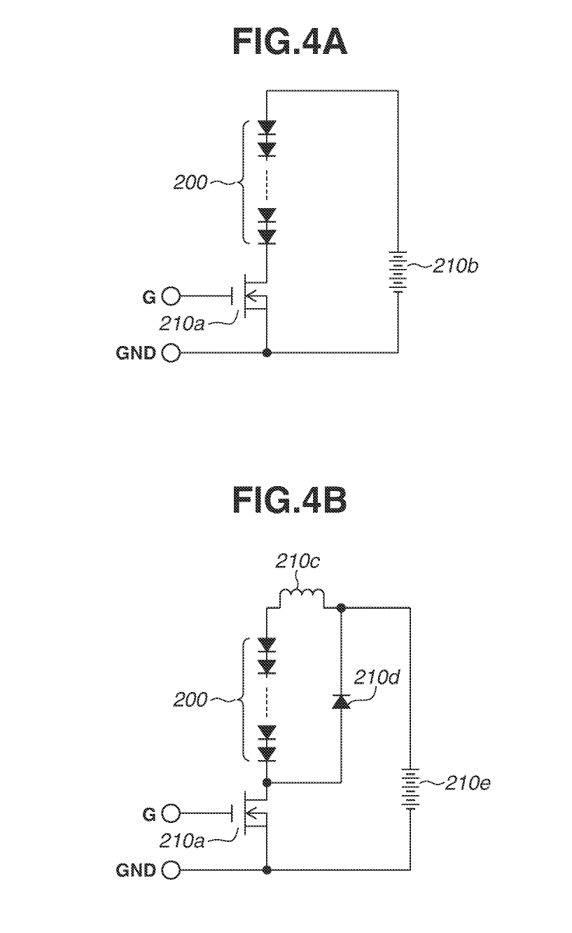

FIGS. 4A and 4B illustrate two different configurations of the drive unit 210 according to the first exemplary embodiment. Either configuration is suitably applicable to the present exemplary embodiment. The configuration of the drive unit 210 illustrated in FIG. 4A will be described below. Referring to FIG. 4A, the light irradiation unit 200 includes a plurality of semiconductor light emission elements (laser diodes) connected in series. A transistor 210a (for example, a metal-oxide-semiconductor field effect transistor (MOSFET)) controls the current flowing in the laser diodes of the light irradiation unit 200. A power source 210b supplies power to the laser diodes of the light irradiation unit 200. In the case of a handheld type probe, the power source 210b for supplying power to the probe 180 may be mounted out of the probe 180 and within the photoacoustic apparatus main body. In this case, it is desirable to mount a bypass capacitor (not illustrated) to reduce the impedance (inductance component) of the power source 210b. The waveform of the current flowing in the laser diodes of the light irradiation unit 200 corresponding to a predetermined optical pulse waveform can be obtained by controlling the gate voltage of the transistor 210a. Implementing the above-described drive unit 210 enables producing the above-described optical pulse waveforms..

FIG. 4B illustrates another configuration of the drive unit 210. Referring to FIG. 4B, the light irradiation unit 200 includes of plurality of semiconductor light emission elements (laser diodes) connected in series. The transistor 210a (for example, a MOSFET) performs ON/OFF control of the current flowing in the laser diodes of the light irradiation unit 200. An inductor 210c is implemented, for example, by a coil or wiring of a printed circuit board. When the transistor 210a turns OFF, a diode 210d. returns the current flowing in the inductor 210c to the inductor 210c. A variable voltage power source 210e supplies power. In the case of a handheld type probe, the variable voltage power source 210e may be mounted out of the probe 180 and within the photoacoustic apparatus main body. In this case, a bypass capacitor (not illustrated) is mounted to reduce the impedance (inductance component) of the power source 210b, The following assumes a case where the transistor 210a turns ON in the configuration illustrated in FIG. 4B. In this case, the inductor 210c is applied with the difference voltage between a total voltage (the sum of the forward voltages of the laser diodes of the light irradiation unit 200, plus the drain-source voltage of the transistor 210a when the transistor 210a turns ON) and the output voltage of the variable voltage power source 210e. The inclination of the current flowing in the inductor 210c can be determined by the voltage applied to the inductor 210c and the inductance of the inductor 210c. More specifically, if the inductance is fixed, the inclination of the current flowing in the inductor 210c can be increased by increasing the voltage of the variable voltage power source 210e. This means that it is possible to control the inclination of the current flowing in the laser diodes of the light irradiation unit 200 and accordingly to control the inclination of the rising waveform of an optical pulse waveform. When the transistor 210a turns OFF, the inductor 210c is applied with the sum of the forward voltages of the laser diodes of the light irradiation unit 200, plus the forward voltage of the diode 210d. This voltage and the inductance of the inductor 210c determine the inclination of the current flowing in the inductor 210c, i.e., the inclination of the falling waveform of an optical pulse waveform. The following assumes a case where each of the drain-source voltage of the transistor 210a when the transistor 210a turns ON and the forward voltage of the diode 210d is sufficiently smaller than the sum of the forward voltages of the laser diodes of the light irradiation unit 200. In this case, it is possible to set the output voltage of the variable voltage power source 210e to a voltage twice the sum of the forward voltages of the laser diodes of the light irradiation unit 200. This setting enables providing the same inclination of the rising and falling waveforms of the current flowing in the laser diodes of the light irradiation unit 200 and accordingly providing the same inclination of the rising and falling waveforms of an optical pulse waveform. In addition, connecting the anode of the diode 210d to the other electrode of the inductor 210c enables further reducing the falling time of the current flowing in the laser diodes of the light irradiation unit 200 when the transistor 210a is turned OFF. In this way, it is possible to obtain a predetermined optical pulse waveform by using the inductor 210c and the variable voltage power source 210e.

### (Reception Unit 120)

The reception unit 120 includes transducers for receiving photoacoustic waves generated in light emission of the light irradiation unit 200 and outputting electrical signals, and a supporting member for supporting the transducers. Examples of components applicable to transducers include piezoelectric materials, capacitive transducers, and transducers using a Fabry-Perot interferometer. Examples of piezoelectric materials include piezoelectric ceramic materials, such as titanic acid lead zirconate (PZT), and macromolecule piezoelectric film materials, such as polyvinylidene fluoride (PVDF). In addition, capacitive transducers may be referred to as Capacitive Micro-machined Ultrasonic Transducers (CMUTs).

An electrical signal acquired by the transducers in each light emission by the light irradiation unit 200 is a time-resolved signal. Therefore, the amplitude of the electrical signal represents a value based on the sound pressure received by the transducers at each time (for example, a value proportional to the sound pressure). It is desirable that transducers are capable of detecting frequency components (typically, ranging from 100 kHz to 10 MHz) of a photoacoustic wave. It is also desirable to arrange a plurality of transducers side by side on a supporting member to form a plane or curved surface called a 1D array, 1.5D array, 1.75D array, or 2D array. FIG. 3 schematically illustrates examples of transducers in a 1D array.

The reception unit 120 may include an amplifier for amplifying time-series analog signals output from the transducers. The reception unit 120 may include an analog-to-digital (A/D) converter for converting time-series analog signals output from the transducers into time-series digital signals. More specifically, the reception unit 120 may include the signal collection unit 140.

To detect acoustic waves from various angles to improve the image accuracy, it is desirable to arrange transducers such that the transducers surround the subject 100 from the entire circumference. If the subject 100 is too large to be surrounded by the transducers from the entire circumference, it is also possible to dispose the transducers on a hemispherical supporting member. The probe 180 including the reception unit 120 having such a shape is suitable not for a handheld type probe but for a mechanical scan type photoacoustic apparatus for moving a probe relatively to the subject 100. To move the probe, a scanning unit such as an XY stage is applicable. The arrangement and the number of transducers and the shape of the supporting member are not limited thereto but may be optimized for the subject 100.

It is desirable to provide a medium for transmitting a photoacoustic wave in a space between the reception unit 120 and the subject 100. This achieves the acoustic impedance matching at the interface between the subject 100 and transducers. Examples of applicable media include water, oil, and ultrasonic gel.

The photoacoustic apparatus 1 may include holding members for holding the subject 100 to stabilize the shape. It is desirable that the holding members have both high light transmissivity and high acoustic wave transmissivity. Examples of applicable holding members include polymethylpentene, polyethylene terephthalate, and acrylics.

When the apparatus according to the present exemplary embodiment generates an ultrasound image in addition to a photoacoustic image through acoustic wave transmission and reception, a transducer may function as a transmission unit for acoustic wave transmission. A transducer as a reception unit and a transducer as a transmission unit may be a single (common) transducer or different transducers.

### (Signal Collection Unit 140)

The signal collection unit 140 includes an amplifier for amplifying an analog electrical signal generated in each light emission of the light irradiation unit 200 and output from the reception unit 120, and an A/D converter for converting the analog signal output from the amplifier into a digital signal. The signal collection unit 140 may include a Field Programmable Gate Array (FPGA) chip.

Operations of the signal collection unit 140 will be described in more detail below. The analog signals output from a plurality of transducers of the reception unit 120 arranged in an array form are amplified by a plurality of corresponding amplifiers and then converted into digital signals by a plurality of corresponding A/D converters. The A/D conversion is performed with an AD conversion rate which is at least twice the bandwidth of the input signal. If the frequency component of a photoacoustic wave is 100 kHz to 10 MHz, as described above, the A/D conversion is performed with an A/D conversion rate (frequency) of 20 MHz or higher, more desirably 40 MHz. The signal collection unit 140 synchronizes the timing of light irradiation with the timing of signal collection processing by using a light emission control signal. More specifically, the signal collection unit 140 starts the A/D conversion with the above-described A/D conversion rate with reference to the light emission time in each light emission of the light irradiation unit 200 and converts the analog signal into a digital signal. As a result, it is possible for each set of a plurality of the transducers to acquire, in each light emission of the light irradiation unit 200, a digital data line at time intervals (A/D conversion intervals) as a reciprocal number of the A/D conversion rate since the light emission time. In this case, the light emission time may be determined with reference to the time when the optical pulse waveform becomes a peak.

The signal collection unit 140 is also referred to as a Data Acquisition System (DAS). According to the present specification, the concept of an electrical signal includes both an analog signal and a digital signal.

As described above, the signal collection unit 140 may be disposed inside the housing 181 of the probe 180. In such a configuration, information is transferred between the probe 180 and the computer 150 as digital signals, resulting in improved noise immunity. The use of high-speed digital signal transmission enables reducing the number of wiring and improving the operability of the probe 180 in comparison with analog signal transmission.

The addition averaging (described below) may also be performed by the signal collection unit 140. In this case, it is desirable to perform addition averaging by using hardware such as a FPGA.

### (Computer 150)

The computer 150 includes an acquisition unit 151, a storage unit 152, and a control unit 153. The acquisition unit 151 in charge of arithmetic/logic operations may include operation circuits including processors such as a CPU and graphics processing unit (GPU), and a FPGA chip. These units may be configured by a single processor or operation circuit or a plurality of processors or operation circuits.

For example, for each of a plurality of transducers, the computer 150 performs combination processing such as the above-described addition averaging of photoacoustic signals acquired in each of a plurality of optical pulse irradiations. The computer 150 also serves as a filter unit for changing the frequency characteristics of a photoacoustic signal when performing processing for weighting the acquired photoacoustic signals in proportion to the intensity of the frequency spectra of photoacoustic waves obtained by the above-described optical pulse waveforms. The filter unit according to the present exemplary embodiment can change the frequency characteristics of an electrical signal based on the peak frequency of an acoustic wave. Examples of applicable filter units include a filter unit having approximately the same characteristics of the frequency spectrum of an acoustic wave and a filter unit for changing the frequency spectrum of an acoustic wave to approximately flat characteristics.

Subsequent exemplary embodiments will be described below centering on a case where simple addition averaging is performed in these pieces of combination processing.

The computer 150 performs addition averaging of data at the same time since the light emission time of the digital data line output from the signal collection unit 140 in each light emission of the light irradiation unit 200. Then, the computer 150 stores the addition-averaged digital data line in the storage unit 152 as an addition-averaged electrical signal (combined photoacoustic signal) resulting from photoacoustic waves. Then, based on the combined photoacoustic signal stored in the storage unit 152, the acquisition unit 151 generates photoacoustic image data (structure images and function images) through image reconstruction and performs other various calculation processing. The acquisition unit 151 may receive various parameter inputs such as the sound speed of the subject 100 and the configuration of a holding unit from the input unit 170 and use them for calculations.

As a reconstruction algorithm used by the acquisition unit 151 to convert an electrical signal into three-dimensional volume data, arbitrary techniques such as a back-projection method in the time domain, a back-projection method in the Fourier domain, and a model base method (repetitive operation method) are applicable. Examples of back projection methods in the time domain include Universal Back-Projection (UBP), Filtered Back-Projection (FBP), and Delay-and-Sum.

The storage unit 152 includes nonvolatile memories such as a random access memory (RAM) and non-transitory storage media such as a read only memory (ROM), magnetic disk, and flash memory. A storage medium storing programs is a non-transitory storage medium. The storage unit 152 includes a plurality of storage media.

The storage unit 152 can store various data including combined photoacoustic signals, photoacoustic image data generated by the acquisition unit 151, and reconstructed image data based on the photoacoustic image data.

The control unit 153 includes an operation element such as a CPU. The control unit 153 controls operations of each component of the photoacoustic apparatus 1. The control unit 153 transmits to the drive unit 210 a light emission control signal and an optical pulse waveform setting signal for a plurality of optical pulses. Then, the laser diodes emit light having specified optical pulse waveforms to irradiate the subject 100 with the light. The control unit 153 also serves as an optical pulse waveform setting unit for setting optical pulse waveforms.

As described above, the control unit 153 controls optical pulse waveforms, the light intensity and the number of emissions of optical pulses, and the gain of photoacoustic signals based on a user instruction or in an automatic way. More specifically, the control unit 153 also serves as a light intensity setting unit for setting the light intensity of each of the at least two different optical pulse waveforms, and also serves as a light emission count setting unit for setting the number of emissions of each of the at least two different optical pulse waveforms.

The control unit 153 also reads a program code stored in the storage unit 152 and controls operations of each component of the photoacoustic apparatus 1. The control unit 153 also adjusts the image on the display unit 160. In this configuration, oxygen saturation distribution images are sequentially displayed during the prove movement and photoacoustic measurement.

The computer 150 may be a workstation specially designed for the present exemplary embodiment. The computer 150 may also be a general-purpose personal computer (PC) or workstation which operates according to program instructions stored in the storage unit 152. Components of the computer 150 may be configured with different hardware components. At least a part of components of the computers 150 may be configured by a single hardware component.

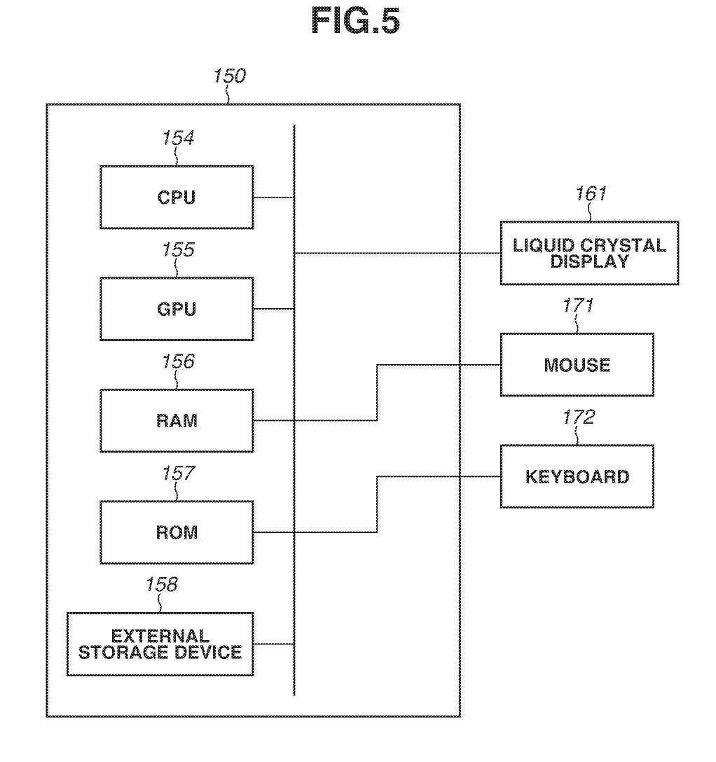

FIG. 5 illustrates an example of a specific configuration of the computer 150 according to the present exemplary embodiment. The computer 150 according to the present exemplary embodiment includes a CPU 154, a GPU 155, a RAM 156, a ROM 157, and an external storage device 158. The computer 150 is connected with a liquid crystal display (LCD) 161 as the display unit 160, and a mouse 171 and a keyboard 172 as the input unit 170.

The computer 150 and the reception unit 120 may be offered in a common housing. A computer provided in the housing may perform a part of signal processing, and a computer provided out of the housing may perform the remaining signal processing. In this case, the computers provided in and out of the housing can be collectively referred to as a computer according to the present exemplary embodiment. More specifically, hardware components of a computer do not need to he stored in one housing. The computer 150 may be implemented by an information processing apparatus which is offered in a cloud computing service and installed at a remote site.

The computer 150 is equivalent to a processing unit of the present exemplary embodiment. In particular, the functions of the processing unit are implemented mainly by the acquisition unit 151.

### (Display Unit 160)

The display unit 160 is a liquid crystal display or an organic electro luminescence (EL) display. The display unit 160 is an apparatus for displaying images and numerical values of a specific position based on the subject information (for example, structure information and function information) acquired by the computer 150. The display unit 160 may display a GUI for operating an image and apparatus. The display unit 160 or the computer 150 may perform image processing (such as luminance value adjustment).

### (Input Unit 170)

The input unit 170 may be an operation console provided with a mouse and a keyboard which can be operated by the user. The display unit 160 may be provided with a touch panel and used as the input unit 170. The input unit 170 receives instructions and numerical inputs from the user and transmits them to the computer 150.

Components of the photoacoustic apparatus 1 may be configured as separate apparatuses or as one integrated apparatus. At least a part of components of the photoacoustic apparatus 1 may be configured as one integrated apparatus.

The control unit 153 of the computer 150 also performs drive control of components included in the photoacoustic apparatus 1. The display unit 160 array display a GUI in addition to images generated by the computer 150. The input unit 170 is configured to allow the user to input information. Using the input unit 170, the user can perform operations such as an operation for starting/ending measurement, an operation for specifying an irradiation mode (described below), and an operation for storing a generated image.

### (Subject 100)

The subject 100 which is not a component of the photoacoustic apparatus 1 will be described below. The photoacoustic apparatus 1 according to the present exemplary embodiment can be used for the purpose of diagnosis and chemical treatment follow-up for malignant tumors and blood vessel diseases of human and animals. Therefore, the subject is assumed to be a diagnosis target portion of a living body, more specifically, breast, internal organs, blood vessel network, head, cervix, abdomen, fingers, and four limbs including fingers and toes. For example, if a human body is a measurement object, then oxyhemoglobin, deoxyhemoglobin, a blood vessel or tumor containing a large amount of oxyhemoglobin or deoxyhemoglobin, or a new blood vessel formed near a tumor may be used as a target optical absorber. Plaque of a carotid wall may also be used as a target optical absorber. When the subject 100 is a human body, the melanin pigment contained in the skin may serve as an optical absorber which causes the above-described disturbance. Pigments such as methylene blue (MB) and indocyanine green (ICG), golden particulates, or a substance introduced from outside as an accumulation or chemical modification of these substances may be used as an optical absorber. A puncture needle or an optical absorber applied thereto may be used as an observation object. The subject 100 may be an inanimate object such as a phantom and a test object.

### (Operation of Exemplary Embodiment)

Then, the subject information acquisition apparatus according to the present exemplary embodiment irradiates the subject 100 with optical pulses having at least two different optical pulse waveforms a plurality of times, receives acoustic waves generated within the subject 100 in light irradiations, and acquires characteristic information about the subject based on the received photoacoustic signals. The first exemplary embodiment will be described in detail below as an exemplary embodiment for alternately emitting two optical pulses having different optical pulse waveforms and performing addition averaging of acquired photoacoustic signals.

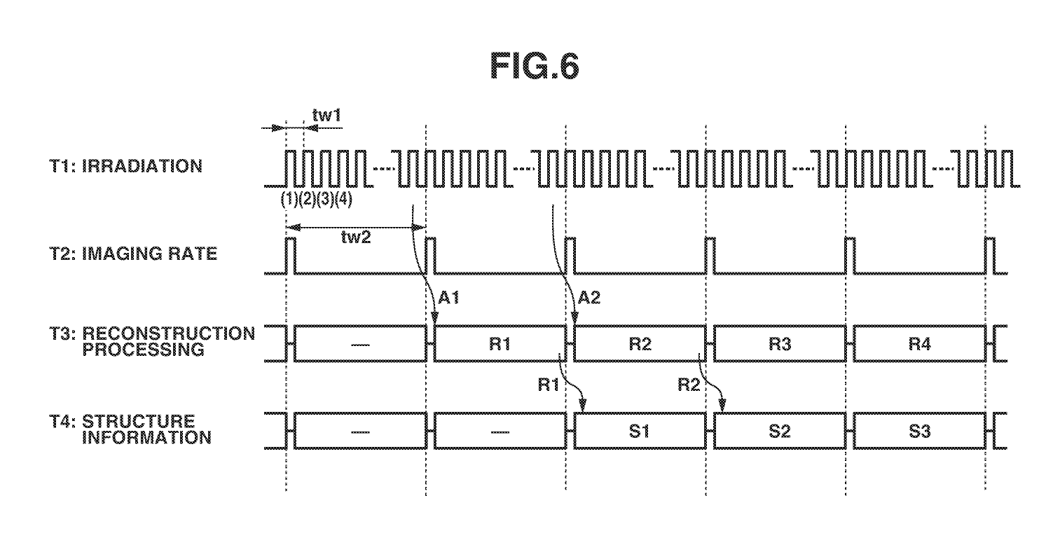

FIG. 6 is a timing diagram illustrating operations according to the first exemplary embodiment in an easy way. Referring to FIG. 6, the horizontal axis indicates time. These operations are controlled by the computer 150, FPGA, or dedicated hardware. A method used by the photoacoustic apparatus 1 according to the present exemplary embodiment to acquire a photoacoustic signal, and a method used by the photoacoustic apparatus 1 to generate a photoacoustic image based on the acquired photoacoustic signal will be described in detail below with reference to FIG.

FIG. 6 is a timing diagram illustrating alternate emissions of optical pulses having two different optical pulse waveforms and the acquisition of structure information based on acquired photoacoustic signals. When blood vessel structure information is acquired, it is desirable to select about 795 nm as the wavelength of the light of the light irradiation unit 200 at which the absorption coefficients of oxyhemoglobin and deoxyhemoglobin are equal.

Since laser diodes have a low light intensity, the photoacoustic apparatus 1 performs the following control for the purpose of improving the S/N ratio. More specifically, the photoacoustic apparatus 1 controls a plurality of semiconductor light emission elements of the light irradiation unit 200 to repetitively emit light at irradiation intervals tw1, as illustrated by T1 in FIG. 6, and acquires photoacoustic signals. The length of the irradiation interval tw1 is set in consideration of Maximum Permissible Exposure (MPE) to the skin. For example, it is desirable to select 0.1 ms as the irradiation interval tw1.

Referring to T1 illustrated in FIG. 6, the light irradiation unit 200 alternately emits optical pulses having the optical pulse waveforms illustrated in FIGS. 1A and 1C, and the acquisition unit 151 acquires photoacoustic signals 168 times ((1) to (4) . . . ) at irradiation intervals tw1, performs addition averaging of the photoacoustic signals, and acquires an addition-averaged photoacoustic signal A1 at imaging frame rate intervals tw2. More specifically, the photoacoustic apparatus 1 performs addition averaging of 83 photoacoustic signals acquired by using the optical pulse waveforms illustrated in FIG. 1A and 83 photoacoustic signals acquired by using the optical pulse waveforms illustrated in FIG. 1C. In this case, the addition averaging is an example and may be other combining processing described above.

Referring to T3 illustrated in FIG. 6, the photoacoustic apparatus 1 performs the above-described reconstruction processing based on the addition-averaged photoacoustic signal A1 to obtain reconstructed image data R1. Then, the reconstructed image data R1 is sequentially output to the display unit 160 at imaging flame rate intervals tw2 (16.6 ms intervals) to be displayed as structure information S1 (refer to T4 illustrated in FIG. 6). The frequency of the imaging frame rate is about 60 Hz.

When the photoacoustic apparatus 1 acquires photoacoustic signals 166 times and performs addition averaging, the frequency of the imaging frame rate is about 60 Hz which coincides with the display frame rate of the display unit 160. If the number of addition averaging differs, the imaging frame rate may not coincide with the display frame rate. In this case, it is desirable to convert the imaging frame rate into a display frame rate by using a frame rate converter (not illustrated) and display data on the display unit 160.

As described above, the photoacoustic apparatus 1 emits optical pulses having at least two different optical pulse waveforms in a plurality of light emissions of the light irradiation unit 200. This enables acquiring information based on the frequency characteristics of a predetermined photoacoustic signal.

The result makes it possible to acquire information about the frequency characteristics of more suitable photoacoustic signals.

Although, in the first exemplary embodiment, the photoacoustic apparatus 1 is configured to alternately emit optical pulses having at least two different optical pulse waveforms, the configuration of the photoacoustic apparatus 1 is not limited thereto. For example, the photoacoustic apparatus 1 may perform control to emit an optical pulse, having the optical pulse waveform illustrated in FIG. 1A, 83 times in succession and subsequently emit an optical pulse, having the optical pulse waveform illustrated in FIG. 1C, 83 times in succession. If the photoacoustic apparatus 1 is configured to alternately emit two different optical pulse waveforms, the time of emission of an optical pulse having the optical pulse waveform illustrated in FIG. 1A is close to the time of emission of an optical pulse having the optical pulse waveform illustrated in FIG. 1C. This makes it possible to reduce the influence of the motion of the subject 100 on the combined portion of photoacoustic signals acquired in light emissions with respective optical pulse waveforms. If the photoacoustic apparatus 1 is configured to continuously emit light, the time of light emissions with respective optical pulse waveforms is reduced, providing an advantage that photoacoustic signals acquired in light emissions with respective optical pulse waveforms are hardly affected by the motion of the subject 100.

### (Modification of First Exemplary Embodiment)

The first exemplary embodiment has been described above centering on an example where photoacoustic signals acquired in emissions of optical pulses having respective optical pulse waveforms are addition-averaged. As described above, it is also possible to change the number of emissions of optical pulses having respective optical pulse waveforms and perform addition averaging of acquired photoacoustic signals. It is also possible to change the light intensity of optical pulses having respective optical pulse waveforms and perform addition averaging of acquired photoacoustic signals. It is also possible to change the number of emissions of optical pulses having respective optical pulse waveforms from the semiconductor light emission elements and perform addition averaging of acquired photoacoustic signals. It is also possible to change the gain for amplifying photoacoustic signals in synchronization with emissions of optical pulses having respective optical pulse waveforms and perform addition averaging of amplified photoacoustic signals. It is also possible to weight the acquired photoacoustic signals in proportion to the intensity of the frequency spectra of photoacoustic waves obtained in light emissions with optical pulse waveforms and perform addition averaging of photoacoustic signals. It is also possible to, before performing addition averaging, flatten the frequency spectra of the obtained photoacoustic waves by using a filter having characteristics for providing a flat frequency spectrum of a photoacoustic wave obtained in light emission with an optical pulse waveform.

As another exemplary embodiment, when performing processing for obtaining reconstructed image data, it is also possible to acquire reconstructed image data by performing the reconstruction processing for each optical pulse, instead of the above-described processing on photoacoustic signals, and then performing the above-described combining processing on the reconstructed image data. However, performing this combining processing on the reconstructed image data includes performing the reconstruction processing in each light emission, which resulting in an increase in the amount of calculations as a drawback.

A plurality of wavelengths may be used as the wavelength of light emitted by the light irradiation unit 200. When a plurality of wavelengths is used, for example, the oxygen saturation as function information can be calculated. According to the present exemplary embodiment, it is possible to, for each imaging frame rate, acquire photoacoustic signals by alternately switching between two different wavelengths, calculate reconstructed image data, and further calculate the oxygen saturation based on the reconstructed image data calculated for the two imaging frame rates. The calculation of the oxygen saturation is discussed in detail in Japanese Patent Application Laid-Open No. 2015-142740. When acquiring the structure information, the photoacoustic apparatus 1 sequentially irradiates the subject 100 with light having at least two different optical pulse waveforms according to the present exemplary embodiment and obtains information about wide frequency characteristics. Then, optical pulse waveforms with other wavelengths for acquiring function information may have a shape of one optical pulse waveform with a long pulse width. This configuration makes it possible to clearly detect structure information regardless of the thickness of a blood vessel and acquire function information free from high-frequency components. Masking the function information with the structure information enables providing detailed information.

A plurality of the exemplary embodiments of the present exemplary embodiment may be implemented in one photoacoustic apparatus and selectively used. Further, the photoacoustic apparatus 1 according to the present exemplary embodiment may be additionally provided with a function of transmitting ultrasonic waves from transducers and performing measurement by using reflection waves. In this case, naturally, the light irradiation unit 200 does not emit light.

The photoacoustic apparatus 1 according to the first exemplary embodiment makes it possible to widen the frequency bandwidth of acoustic waves to be detected by irradiating a plurality of times the subject 100 with pulsed light at least a part of which have different pulse waveforms. This makes it possible to acquire information about substances ranging from a large-sized substance corresponding to a low-frequency to a small-sized substance corresponding to a high-frequency out of optical absorbers distributed over the subject 100. In addition, the frequency bandwidth of acoustic waves to be detected can be further widened by increasing the number of types of pulse waveforms.

A second exemplary embodiment will be described below centering on a photoacoustic apparatus, as an example of a subject information acquisition apparatus, for irradiating a subject with pulsed light a plurality of times, receiving photoacoustic waves from the subject, and acquiring intra-subject information such as blood vessel image (structure image).

The present exemplary embodiment is intended to simultaneously emit light having at least two different optical pulse waveforms in a plurality of light emissions to irradiate the subject 100 with the light, and acquire information based on acquired photoacoustic signals. Such a configuration makes it possible to achieve desired frequency spectra of photoacoustic waves generated in light irradiation, i.e., desired frequency characteristics of photoacoustic signals as electrical signals converted from photoacoustic waves. However, the effect of the photoacoustic apparatus 1 according to the present exemplary embodiment is also applicable to the configuration for simultaneously emitting light having at least two different optical pulse waveforms to irradiate once the subject 100 with optical pulses. Although the present exemplary embodiment focuses on a photoacoustic apparatus having a handheld type probe, the present exemplary embodiment is also applicable to a photoacoustic apparatus having a probe on a stage for mechanical scanning. The present exemplary embodiment will be described below centering on a photoacoustic apparatus having a handheld type probe that includes a plurality of semiconductor light emission elements and that is wired to the apparatus main body. However, the present exemplary embodiment is also applicable to a photoacoustic apparatus having a handheld type probe, having a power source such as a battery, for wirelessly transmitting photoacoustic signals of the handheld type probe to the photoacoustic apparatus main body. If the handheld type probe has the functions of the present exemplary embodiment as described above, the photoacoustic apparatus according to the present exemplary embodiment means the handheld type probe itself.

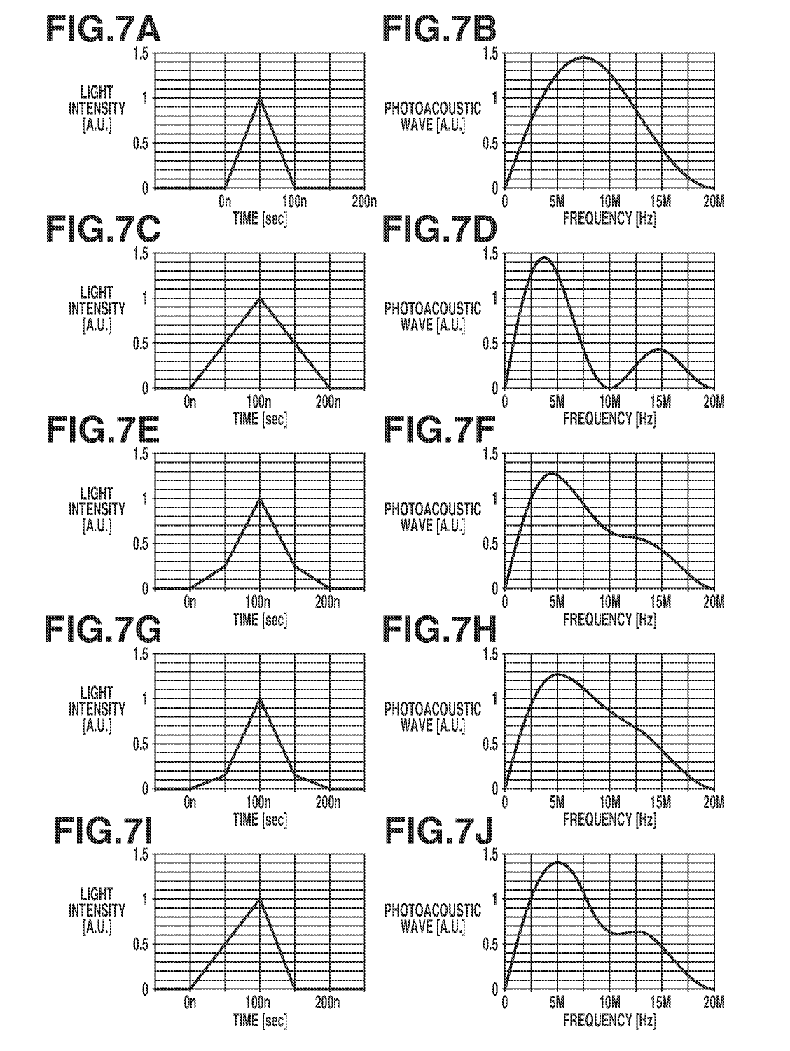

The basic concept of the present exemplary embodiment will be described below. Reference numerals used in the descriptions of the second exemplary embodiment correspond to those illustrated in FIGS. 7A to 12.

FIGS. 7A to 7J are graphs illustrating relations between optical pulse waveforms and photoacoustic signals for describing the basic concept of the present exemplary embodiment. FIGS. 7A and 7C are graphs illustrating optical pulse waveforms, where the vertical axis indicates the light intensity and the horizontal axis indicates time. FIGS. 7B and 7D are graphs illustrating simulations of the frequency spectrum of photoacoustic waves obtained when the subject 100 is irradiated with the optical pulse waveforms illustrated in FIGS. 1A and 1C, respectively. The vertical axis indicates the photoacoustic wave intensity and the horizontal axis indicates the frequency. When the subject 100 is irradiated with an optical pulse having a short pulse width (100 ns), as illustrated in FIG. 7A, the frequency spectrumpeak value of the obtained photoacoustic wave is about 7.5 MHz, as illustrated in FIG. 7B. On the other hand, when the subject 100 is irradiated with an optical pulse having a long pulse width (200 ns), as illustrated in FIG. 7C, the frequency spectrum peak value of the obtained photoacoustic wave is about 3.75 MHz, as illustrated in FIG. 7D.

According to the first exemplary embodiment, the photoacoustic apparatus 1 acquires information about photoacoustic signals based on such photoacoustic signals obtained by simultaneously irradiating the subject 100 with optical pulses having different optical pulse waveforms. For example, a light irradiation unit including 12 semiconductor light emission elements controls six light emission elements to emit an optical pulse having the optical pulse waveform illustrating in FIG. 7A and controls another six light emission elements to simultaneously emit an optical pulse having the optical pulse waveform illustrated in FIG. 7C to irradiate the subject 100 with these optical pulses. The result makes it possible to obtain a photoacoustic signal having the frequency spectrum of a photoacoustic wave as a result of adding the frequency spectra of the photoacoustic waves illustrated in FIGS. 7B and 7D. More specifically, FIG. 7E illustrates an optical pulse waveform obtained by simultaneously emitting optical pulses having the optical pulse waveforms illustrated in FIGS. 7A and 7C. “Simultaneous emission” means emission of optical pulse waveforms with synchronized peak time. Referring to FIG. 7E, the vertical axis indicates the light intensity and the horizontal axis indicates time. FIG. 7F is a graph illustrating a simulation of the frequency spectrum of a photoacoustic wave obtained when the subject 100 is irradiated with the optical pulse waveform illustrated in FIG. 1E. The vertical axis indicates the photoacoustic wave intensity and the horizontal axis indicates the frequency. As illustrated in FIG. 7F, when the optical pulse waveforms illustrated in FIGS. 7A and 7C are simultaneously emitted to irradiate the subject 100, the peak frequency of the photoacoustic wave is about 4.25 MHz, and the −6 dB bandwidth ranges from about 1.25 to about 10 MHz. The characteristics illustrated in FIG. 7F provide a wider range of high-frequency components than the characteristics illustrated in FIG. 7D where the peak frequency of the photoacoustic wave is about 3.75 MHz and the −6 dB bandwidth ranges from about 1.25 to about 6.25 MHz. Meanwhile, the peak frequency of the photoacoustic wave illustrated in FIG. 7B is about 7.5 MHz, and the −6 dB bandwidth ranges from about 2 to about 12.75 MHz. Such frequency characteristics provide a sufficiently wide range of high-frequency components but low levels of low-frequency components, which possibly degrade the visibility of a blood vessel with a thickness of 0.75 mm or more.

For example, another optical pulse waveform having a pulse width of 300 nm (not illustrated) may be simultaneously emitted. For example, a light irradiation unit including 12 semiconductor light emission elements controls four light emission elements to emit an optical pulse having the optical pulse waveform illustrating in FIG. 7A, controls another four light emission elements to simultaneously emit an optical pulse having the optical pulse waveform illustrated in FIG. 7C, and controls yet another four light emission elements to simultaneously emit an optical pulse having a pulse width of 300 nm (not illustrated) to irradiate the subject 100 with these optical pulses. Emitting light in this way enables further improving the intensity of low-frequency components of the photoacoustic wave.

As another method, controlling the light intensity enables easily changing the frequency spectrum of a photoacoustic wave. For example, a light irradiation unit including 12 semiconductor light emission elements controls six light emission elements to emit an optical pulse having the optical pulse waveform illustrating in FIG. 7A and controls another six light emission elements to simultaneously emit an optical pulse having a light intensity twice that of the optical pulse waveform illustrated in FIG. 7C to irradiate the subject 100 with these optical pulses. This optical pulse waveform is illustrated in FIG. 7G. The result makes it possible to obtain a combined photoacoustic signal representing the frequency spectrum of a photoacoustic wave as a result of adding the frequency spectra of the photoacoustic waves illustrated in FIGS. 7B and 7D, weighted with a ratio of 1:2, respectively. The frequency spectrum of the photoacoustic wave is illustrated in FIG. 7H. In this case, the subject 100 is irradiated with a high light intensity of an optical pulse having the optical pulse waveform illustrated in FIG. 1C. Therefore, it is possible to further increase high-frequency components of the frequency characteristics of the photoacoustic wave illustrated in FIG. 7F. As illustrated in FIG. 7H, the peak frequency of the photoacoustic wave is about 5.25 MHz, and the −6 dB bandwidth ranges from about 1.5 to about 12.75 MHz. The characteristics illustrated in FIG. 7H provide a wider range of high-frequency components than the characteristics illustrated in FIG. 7F where the peak frequency of the photoacoustic wave is about 4.25 MHz and the −6 dB bandwidth ranges from about 1.25 to about 10 MHz.

As another method for controlling the light intensity, the light intensity may be controlled by increasing or decreasing the number of semiconductor light emission elements of the light irradiation unit 200 for emitting optical pulses having respective optical pulse waveforms. More specifically, even if each individual semiconductor light emission element emits light with the same light intensity, the light intensity of optical pulses to be radiated onto the subject 100 can be controlled by increasing or decreasing the number of semiconductor light emission elements for emitting light. More specifically, for example, a light irradiation unit including 12 semiconductor light emission elements controls four light emission elements to emit an optical pulse having the optical pulse waveform illustrating in FIG. 7A and controls eight light emission elements to simultaneously emit an optical pulse having a light intensity of the optical pulse waveform illustrated in FIG. 7C to irradiate the subject 100 with these optical pulses. Performing this control method also enables obtaining similar photoacoustic waves to those obtained through control of the semiconductor light emission elements. This control method does not change the light intensity of the semiconductor light emission elements in an analog way, which thus simplifies the circuit configuration.

According to the present exemplary embodiment, as described above, the shapes of the optical pulse waveforms are set so that different frequencies of frequency spectrum peak values of photoacoustic waves are obtained by using optical pulses having at least two different optical pulse waveforms. Such a configuration suitably makes it easier to adjust the frequency spectrum of a photoacoustic wave. To obtain different frequencies of frequency spectrum peak values of photoacoustic waves, it is desirable to set triangle optical pulse waveforms which are different in pulse width. More specifically, it is desirable to select the pulse width of optical pulse waveforms from 100 to 1000 ns. For example, triangle waves having a 100 ns pulse width and a 200 ns pulse width may be used, or triangle waves having a 200 ns pulse width and a 300 ns pulse width may be used. In addition, the above-described three different triangle waves may be used.

An optical pulse having an asymmetrical optical pulse waveform with a rising time of 100 ns and a falling time of 50 ns, as illustrated in FIG. 7I, may be used. In this case, a frequency spectrum of a photoacoustic wave as illustrated in FIG. 7J can be obtained. If such characteristics are taken into consideration, this frequency spectrum is naturally applicable to the present exemplary embodiment. Optical pulse waveforms having any shapes are applicable to the present exemplary embodiment.

As described above, it is possible to control the frequency spectrum of a photoacoustic wave by simultaneously emitting light pulses having at least two different optical pulse waveforms by using a part of a plurality of semiconductor light emission elements of the light irradiation unit 200.

As discussed in Japanese Patent Application Laid-Open No. 2016-47114, the present exemplary embodiment makes it possible to suitably control the frequency spectrum of a photoacoustic wave so that the frequency of a combined optical pulse waveform is close to the frequency at which the reception sensitivity of a detection unit (reception unit) is maximized. Therefore, it is also possible to set the shape and light quantity of optical pulse waveforms to be simultaneously emitted. In the case of a photoacoustic apparatus having an exchangeable probe, it is desirable to control the shape and light quantity of optical pulse waveforms to be simultaneously emitted according to the frequency characteristics of the reception unit of the probe. In addition, it is also possible to set the shape and light quantity of optical pulse waveforms to be simultaneously emitted according to the frequency spectra of the photoacoustic waves determined by the thickness of a blood vessel in the subject region under observation. In addition, it is also possible to control the shape and light quantity of optical pulse waveforms to be simultaneously emitted to enable controlling the frequency spectrum of a photoacoustic wave according to a user instruction. For example, when the user wants to observe a fine blood vessel, the following setting may be made: a pulse width of optical pulses to be simultaneously emitted is set to be short to increase high-frequency components of the frequency spectrum of a photoacoustic wave. When the subject region under observation is deep inside of the subject 100, high-frequency components of a photoacoustic signal are largely attenuated. In this case, therefore, a computer may automatically set the shapes of optical pulse waveforms to be simultaneously emitted to increase high-frequency components of the frequency characteristics of the photoacoustic wave. It is also possible to provide a pressure sensor on the handheld type probe and measure the pressing force with which the probe is pressed onto the subject 100. The computer may change the shapes of photoacoustic waves to be simultaneously emitted depending on the magnitude of the pressing force. For example, when observing details, the user may strongly press the probe onto the subject in an unconscious way. In the case of a large pressing force, a pulse width of optical pulses to be simultaneously emitted is set to short to increase high-frequency components of the frequency spectrum of the photoacoustic wave.

As described above, in a photoacoustic apparatus for acquiring information based on photoacoustic signals generated by pulsed light emissions from the light irradiation units, at least two different optical pulse waveforms are simultaneously emitted in a plurality of pulsed light emissions from the light irradiation units. Emitting light in this way enables acquiring information based on the desired frequency characteristics of a photoacoustic signal. The following describes the present exemplary embodiment in which optical pulses having three or more different pulse waveforms are simultaneously emitted a plurality of times, and acquired photoacoustic signals are addition-averaged. The present exemplary embodiment is also applicable to above-described other configurations of light emission control.

### (Configuration of Photoacoustic Apparatus)

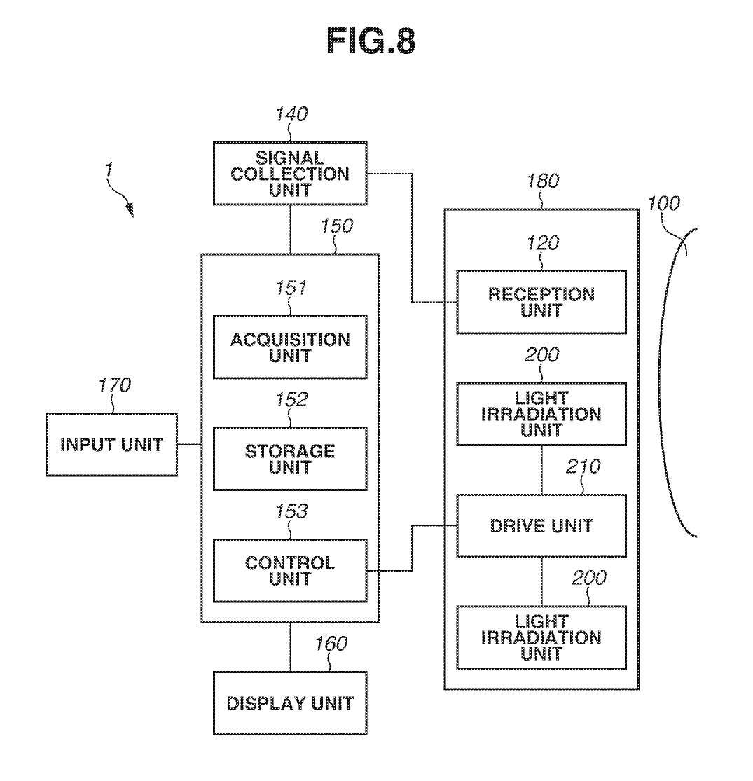

FIG. 8 is a block diagram illustrating the photoacoustic apparatus 1 according to the present exemplary embodiment. Referring to the block diagram illustrated in FIG. 8, the photoacoustic apparatus 1 according to the present exemplary embodiment includes a plurality of the light irradiation units 200 for irradiating the subject 100 with pulsed light, and the control unit 153 for controlling a plurality of the light irradiation units 200 to perform approximately simultaneous radiations of optical pulses to irradiate the subject 100 with the optical pulses. “Approximately simultaneous radiations” refer not only to radiations of optical pulses to the subject 100 at the same time but also to radiations thereof at different timing to such an extent that the effect of the present exemplary embodiment can be obtained.

The photoacoustic apparatus 1 further includes a reception unit 120 for receiving acoustic waves generated when the subject 100 is irradiated with optical pulses and converting the acoustic waves into electrical signals, and an acquisition unit 151 for acquiring information about the subject 100 based on the converted electrical signals. The control unit 153 controls the light irradiation units 200 so that optical pulses to be emitted from a plurality of the light irradiation units 200 have mutually different pulse waveforms. As described above, since optical pulses to be radiated from a plurality of the light irradiation units 200 onto the subject 100 have mutually different pulse waveforms, acoustic waves generated by the optical pulses are mutually different in frequency. Therefore, the frequency bandwidth of the detected acoustic waves can be widened by increasing the number of types of pulse waveforms of optical pulses to be radiated onto the subject 100. The result makes it possible to acquire information about substances ranging from a large-sized substance corresponding to a low-frequency acoustic wave to a small-sized substance corresponding to a high-frequency acoustic wave out of optical absorbers distributed over the subject 100.

Although, in the present exemplary embodiment, different pulse waveforms typically mean different pulse widths and are not limited thereto.

According to the present exemplary embodiment, the control unit 153 may control the light irradiation units 200 so that the optical pulses emitted from a plurality of the light irradiation units 200 are mutually different in peak intensity.

The light irradiation units 200 may include a plurality of semiconductor light emission elements. In this case, the control unit 153 may control the peak intensity of pulsed light either by controlling the luminescence intensity of the light emission elements or by controlling the number of light emission elements to be driven to emit light out of a plurality of the light emission elements.

The photoacoustic apparatus 1 according to the present exemplary embodiment includes a probe 180, a signal collection unit 140, a computer 150, a display unit 160, and an input unit 170. The probe 180 includes light irradiation units 200, a drive unit 210, and a reception unit 120. The computer 150 includes an acquisition unit 151, a storage unit 152, and a control unit 153. Portions connected with cables, other than the probe 180, are also collectively referred to as a photoacoustic apparatus main body.

According to the present exemplary embodiment, the light irradiation units 200 may include the light source unit. The light source unit may include a plurality of semiconductor light emission elements. To improve the S/N radio of photoacoustic signals, the light irradiation units 200 emit light a plurality of times, and the acquisition unit 151 acquires photoacoustic signals and performs addition averaging of photoacoustic signals. To achieve the effect of the present exemplary embodiment, the semiconductor light emission elements may emit light only once. The drive unit 210 drives a plurality of the semiconductor light emission elements of the light irradiation units 200 to emit light having at least two different optical pulse waveforms. The drive unit 210 controls the optical pulse waveform of the light irradiation unit 200 for each semiconductor light emission element in light emission. The optical pulse waveform may be desirably determined as described above.

A plurality of the semiconductor light emission elements of the light irradiation unit 200 is divided into at least two different groups of optical pulse waveforms, and the drive unit 210 drives these Tight emission elements to simultaneously emit light. Then, the light irradiation unit 200 simultaneously irradiates the subject 100 with light having at least two different optical pulse waveforms. The reception unit 120 receives photoacoustic waves generated in the subject 100 in light emissions by a plurality of the semiconductor light emission elements and outputs an analog electrical signal (photoacoustic signal). The signal collection unit 140 converts the analog signal output from the reception unit 120 in each light emission into a digital signal and outputs the digital signal to the computer 150.

The computer 150 combines the digital signals output from the signal collection unit 140 in respective light emissions by using the acquisition unit 151, the storage unit 152, and the control unit 153 and stores the resultant signal in the storage unit 152 as an electrical signal (combined photoacoustic signal) resulting from photoacoustic waves. To improve the S/N ratio, it is desirable to emit optical pulses a plurality of times and perform addition averaging.

The computer 150 performs such processing as image reconstruction on the digital signal stored in the storage unit 152 to generate photoacoustic image data. Then, the photoacoustic image data is displayed on the display unit 160.

The computer 150 also controls operations of the entire photoacoustic apparatus 1 and performs optical pulse waveform setting and light emission control.

The computer 150 may perform image processing for display on the obtained photoacoustic image and processing for combining graphics data for graphical user interfaces (GUIs, not illustrated).

The user (doctor or engineer) can perform diagnosis by checking a photoacoustic image displayed on the display unit 160. Based on a storage instruction from the user or the computer 150, the display image may be stored in a memory of the computer 150 or in a data management system connected with the photoacoustic apparatus 1 via a network. The input unit 170 receives an instruction from the user.

### (Detailed Configuration of Each Block)

A desirable configuration of each block will be described in detail below.

### (Probe 180)

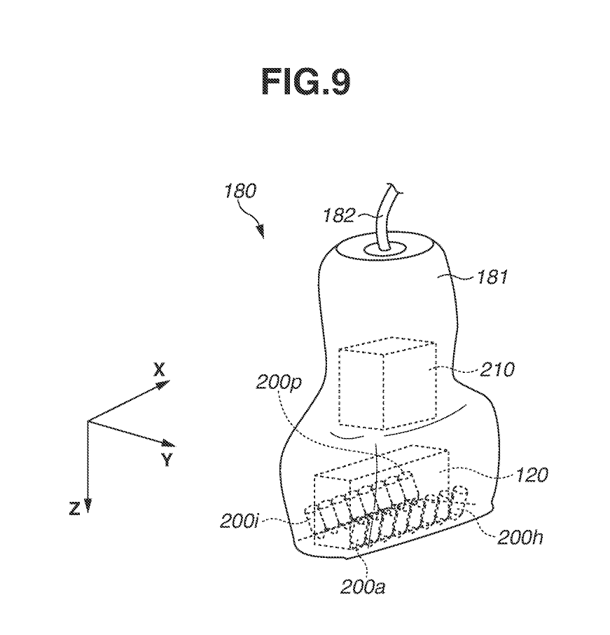

FIG. 9 illustrates a structure of the probe 180 according to the first exemplary embodiment.

Referring to FIG. 9, the probe 180 includes light irradiation units 200, a drive unit 210, a reception unit 120, and a housing 181. The housing 181 surrounds the light irradiation units 200, the drive unit 210, and the reception unit 120. Grasping the housing 181, the user can use the probe 180 as a handheld type probe.

The light irradiation units 200 irradiate the subject 100 with optical pulses. As described above, the drive unit 210 is controlled by the light irradiation units 200 to emit an optical pulse having a desired optical pulse waveform in each optical pulse emission.

The X, Y, and Z axes illustrated in FIG. 9 indicate coordinate axes of when the probe 180 is statically placed and do not limit the direction when the probe 180 is being used.

The probe 180 illustrated in FIG. 9 is connected with the signal collection unit 140 via a cable 182. The cable 182 includes a wiring for supplying power to the light irradiation units 200, a wiring for a light emission control signal, and a wiring for outputting the analog signal from the reception unit 120 to the signal collection unit 140. A connector may be provided on the cable 182 to separate the probe 180 from other components of the photoacoustic apparatus 1.

### (Light Irradiation Unit 200)

The light irradiation unit 200 according to the present exemplary embodiment irradiates the subject 100 with pulsed light generated by the light source.

The light irradiation unit 200 includes, for example, 16 semiconductor light emission elements such as laser diodes. The semiconductor light emission elements are not limited to LDs and may be LEDs. The type and the number of semiconductor light emission elements are determined by the specified light intensity. Eight laser diodes (200a to 200h) and another eight laser diodes (200i to 200p) are disposed to face each other across the reception unit 120. These laser diodes irradiate the subject 100 with light. Each laser diode is mounted toward the maximum sensitivity direction of the reception unit 120. More specifically, as illustrated in FIG. 9, the laser diodes 200a to 200h and the laser diodes 200i to 200p are mounted in such a manner that the laser diodes 200a to 200h and the laser diodes 200i to 200p are inclined toward the direction of the reception unit 120. Although, in FIG. 9, the reception unit 120 is mounted between two sets of the laser diodes, light sources may be collectively disposed on one side. In addition, more than 16 laser diodes may be used. Although, in FIG. 9, discrete parts are arranged, a plurality of dies cut out from a semiconductor wafer may be mounted on a metal base or printed circuit board. The arrangement of the laser diodes is not limited to the above-described arrangement. The laser diodes may be mounted in any arrangement depending on the probe shape or other conditions as long as the subject 100 can be favorably irradiated with optical pulses.

For example, it is desirable that the pulse width of light emitted from the light irradiation unit 200 is 10 ns or more and 1 us or less, more desirably, 100 ns or more and 800 ns or less. Although the wavelength of the light of the light irradiation unit 200 is desirably 400 nm or more and 1600 nm or less, it is desirable to determine the wavelength according to the optical absorption characteristics of the optical absorber subjected to imaging. When imaging a blood vessel with a high resolution, light having a wavelength (400 or more and 800 nm or less) which is largely absorbed in a blood vessel may be used. When imaging a deep inside of a living body, light having a wavelength (700 nm or more and 1100 nm or less) which is absorbed little in a background tissue (water, fat, etc.) of a living body may be used. According to the present exemplary embodiment, a desired wavelength is set to 975 nm which allows acquisition of blood vessel structure information and is reachable to deep portions of the subject 100. A photoacoustic wave equivalent to that acquired through the emission of the desired wavelength can be acquired by simultaneously irradiating the subject 100 with light of a plurality of light sources having other wavelengths satisfying the above-described condition.

### (Driving Unit 210)

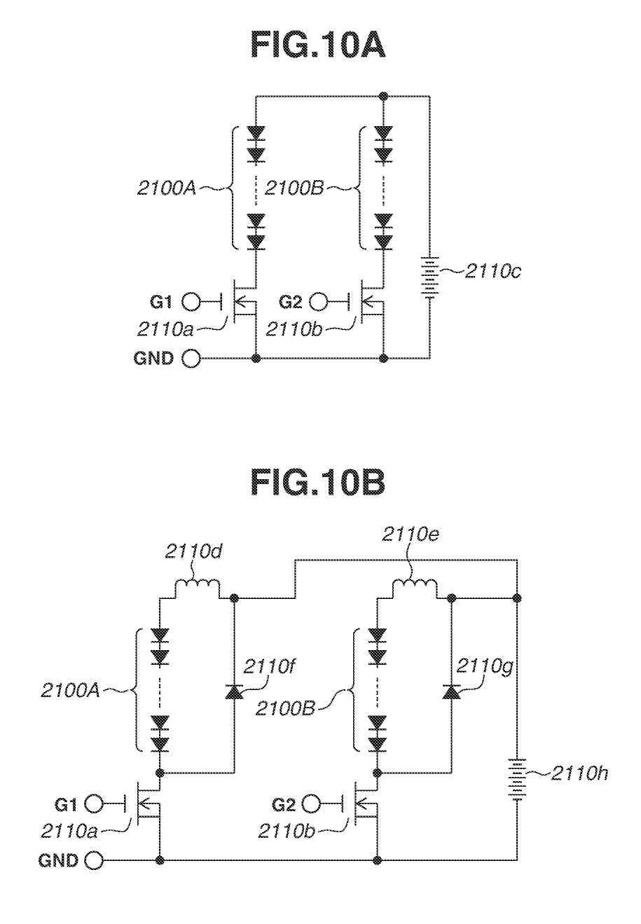

FIGS. 10A and 10E illustrate two different configurations of the drive unit 210 according to the first exemplary embodiment. Either configuration is suitably applicable to the present exemplary embodiment. The configuration of the drive unit 210 illustrated in FIG. 10A will be described below. Referring to FIG. 10.A, the light irradiation unit 200 includes two sets of a plurality of serially-connected semiconductor light emission elements (laser diodes) 2100A and 2100B. For example, a plurality of the serially-connected semiconductor light emission elements 2100A corresponds to eight laser diodes (2100a to 2100h), and a plurality of the serially-connected semiconductor light emission elements 2100B corresponds to another eight laser diodes (2100i to 2100p). In another configuration, a plurality of the serially-connected semiconductor light emission elements 2100A and a plurality of the serially-connected semiconductor light emission elements 2100B may face each other across the reception unit 120. In the former case (FIG. 10A), wiring between a plurality of serially-connected semiconductor light emission elements can be made in the shortest distance, providing an advantage that the inductive component can be reduced as much as possible. In the latter case (FIG. 10B), there is an advantage that optical pulses having respective optical pulse waveforms can be almost uniformly radiated onto the subject 100.

The transistors 2110a and 2110b (for example, MOSFETs) control the currents flowing in a plurality of the serially-connected semiconductor light emission elements 2100A and 2100B, respectively, in the light irradiation unit 200 with different pulse widths. A power source 2110c supplies power to the semiconductor light emission elements of the light irradiation unit 200. In the case of a handheld type probe, the power source 2110c for supplying power to the probe 180 may be mounted out of the probe 180 and within the photoacoustic apparatus main body. In this case, it is desirable to mount a bypass capacitor (not illustrated) to reduce the impedance (inductive component') of the power source 2110c. Controlling the gate voltage of the transistors 2110a and 2110b enables obtaining two different current waveforms flowing in a plurality of the serially-connected semiconductor light emission elements 2100A and 2100B, respectively, in the light irradiation unit 200 corresponding to a desired optical pulse waveform. Implementing the above-described drive unit 2110 enables implementing simultaneous light emissions with the at least two different optical pulse waveforms.

Another configuration of the drive unit 210 is illustrated in FIG. 10B. Referring to FIG. 10B, the light irradiation unit 200 includes two sets of a plurality of serially-connected semiconductor light emission elements (laser diodes) 2100A and 2100B.

The transistors 2110a and 2110b (for example, MOSFETs) perform ON/OFF control of the currents flowing in a plurality of the serially-connected semiconductor light emission elements 2100A and 2100B, respectively, in the light irradiation unit 200 with different pulse widths.

Inductors 2110d and 2110e are implemented, for example, by using coils or wirings on a printed circuit board. When a plurality of the serially-connected semiconductor light emission elements 2100A and 2100B are controlled with different pulse widths, the inductors 2110d and 2110e are designed to have different inductances, respectively. When reducing the pulse width, it is desirable to reduce inductance. When the transistor 2110a turns OFF, a diode 2110f returns the current flowing in the inductor 2110d. to the inductor 2110d, Likewise, when the transistor 2110b turns OFF, a diode 2110g returns the current flowing in the inductor 2110e to the inductor 2110e. A variable voltage power source 2110h supplies power. When flexibility is provided through different pulse width settings, there are provided two different variable voltage power sources 2110h to which voltages can be independently set. In the case of a handheld type probe, the variable voltage power source 2110h may be mounted out of the probe 180 and within the photoacoustic apparatus main body. In this case, a bypass capacitor (not illustrated) is mounted to reduce the impedance (inductive component) of the variable voltage power source 2110h. The following description is given of a case where the transistor 2110a turns ON in the configuration illustrated in FIG. 10B. The inductor 2110d is applied with the difference voltage between a total voltage (the sum of the forward voltages of a plurality of the serially-connected semiconductor light emission elements 2100A, plus the drain-source voltage of the transistor 2110a when ON) and the output voltage of the variable voltage power source 2110h, The inclination of the current flowing in the inductor 2110d can be determined by the voltage applied to the inductor 2110d and the inductance of the inductor 2110d, More specifically, if the inductance is fixed, the inclination of the current flowing in the inductor 2110d. can be increased by increasing the voltage of the variable voltage power source 2110h. This means that it is possible to control the inclination of the current flowing in a plurality of the serially-connected semiconductor light emission elements 2100A in the light irradiation unit 200 and accordingly to control the rising waveform of an optical pulse waveform. When the transistor 2110a turns OFF, the inductor 2110d is applied with the sum of the forward voltages of a plurality of the serially-connected semiconductor light emission elements 2100A, plus the forward voltage of the diode 210f. This voltage and the inductance of the inductor 2110d determine the inclination of the current flowing in the inductor 2110d and accordingly determine the falling waveform of an optical pulse waveform. The following description is given of an example case where each of the drain-source voltage of the transistor 2110a. when ON and the forward voltage of the diode 2110f is sufficiently smaller than the sum of the forward voltages of a plurality of the serially-connected semiconductor light emission elements 2100A. In this case, it is desirable to set the output voltage of the variable voltage power source 2110h to a voltage twice the sum of the forward voltages of a plurality of the serially-connected semiconductor light emission elements 2100A. This setting enables providing the same inclination of the rising and falling waveforms of the current flowing in a plurality of the serially-connected semiconductor light emission elements 2100A and accordingly providing approximately the same inclination of the rising and falling waveforms of an optical pulse waveform. In addition, connecting the anode of the diode 2110f to the other electrode of the inductor 2110d enables reducing the falling time of the current flowing in a plurality of the serially-connected semiconductor light emission elements 2100A when the transistor 2110a is turned OFF. In this way, it is possible to obtain a desired optical pulse waveform by using the inductor 2110d and the variable voltage power source 2110h.

Likewise, it is also possible to desirably control the inclination of the rising and falling waveforms of the current flowing in a plurality of the serially-connected semiconductor light emission elements 2100B in the light irradiation unit 200 and accordingly the optical pulse waveform by using the inductors 2110e and the variable voltage power source 2110h.

### (Reception Unit 120)

The reception unit 120 includes transducers for receiving photoacoustic waves generated in light emission of the light irradiation unit 200 and outputting electrical signals, and a supporting member for supporting the transducers. Examples of component applicable to transducers include piezoelectric materials, capacitive transducers, and transducers using a Fabry-Perot interferometer. Examples of piezoelectric materials include piezoelectric ceramic materials such as titanic acid lead zirconate (PZT) and macromolecule piezoelectric film materials such as polyvinylidene fluoride (PVDF). In addition, capacitive transducers may be referred to as Capacitive Micro-machined Ultrasonic Transducers (CMUTs).

An electrical signal acquired by the transducers in each light emission by the light irradiation unit 200 is a time-resolved signal. Therefore, the amplitude of the electrical signal represents a value based on the sound pressure received by the transducers at each time (for example, a value proportional to the sound pressure). It is desirable that transducers are capable of detecting frequency components (typically, ranging from 100 kHz to 10 MHz) of a photoacoustic wave. It is also desirable to arrange a plurality of transducers side by side on a supporting member to form a plane or curved surface called a 1D array, 1.5D array, 1.75D array, or 2D array. FIG. 9 schematically illustrates examples of transducers in a 1D array.

The reception unit 120 may include an amplifier for amplifying time-series analog signals output from the transducers. The reception unit 120 may include an A/D converter for converting time-series analog signals output from the transducers into time-series digital signals. More specifically, the reception unit 120 may include the signal collection unit 140.

To detect acoustic waves from various angles to improve the image accuracy, it is desirable to arrange the transducers to surround the subject 100 from the entire circumference. If the subject 100 is too large to be surrounded by the transducers from the entire circumference, it is also possible to dispose the transducers on a hemispherical supporting member. The probe 180 including the reception unit 120 having such a shape is suitable not for a handheld type probe but for a mechanical scan type photoacoustic apparatus for moving a probe relatively to the subject 100. To move the probe, a scanning unit such as an XY stage is applicable. The arrangement and thee number of transducers, and the shape of the supporting member are not limited thereto but may be optimized for the subject 100.

It is desirable to provide a medium for transmitting a photoacoustic wave in a space between the reception unit 120 and the subject 100. This achieves the acoustic impedance matching at the interface between the subject 100 and transducers. Examples of applicable media include water, oil, and ultrasonic gel.

The photoacoustic apparatus 1 may include holding members for holding the subject 100 to stabilize the shape. It is desirable that the holding members have both high light transmissivity and high acoustic wave transmissivity. Examples of applicable holding members include polymethylpentene, polyethylene terephthalate, and acrylics.

When the apparatus according to the present exemplary embodiment generates an ultrasound image in addition to a photoacoustic image through acoustic wave transmission and reception, a transducer may function as a transmission unit for acoustic wave transmission. A transducer as a reception unit and a transducer as a transmission unit may be a single (common) transducer or different transducers.

### (Signal Collection Unit 140)

The signal collection unit 140 includes an amplifier for amplifying an analog electrical signal generated in each light emission of the light irradiation unit 200 and output from the reception unit 120, and an A/D converter for converting the analog signal output from the amplifier into a digital signal. The signal collection unit 140 may include a Field Programmable Gate Array (FPGA) chip.

Operations of the signal collection unit 140 will be described in more detail below. The analog signals output from a plurality of transducers of the reception unit 120 arranged in an array form are amplified by a plurality of corresponding amplifiers and then converted into digital signals by a plurality of corresponding A/D converters. The A/D conversion is performed with an A/D conversion rate which is at least twice the bandwidth of the input signal. If the frequency component of a photoacoustic wave is 100 kHz to 10 MHz, as described above, the A/D conversion is performed with an A/D conversion rate (frequency) of 20 MHz or higher, more desirably 40 MHz. The signal collection unit 140 synchronizes the timing of light irradiation with the timing of signal collection processing by using a light emission control signal. More specifically, the signal collection unit 140 starts the A/D conversion with the above-described A/D conversion rate with reference to the light emission time in each light emission of the light irradiation unit 200 and converts the analog signal into a digital signal. As a result, it is possible for each set of a plurality of the transducers to acquire, in each light emission of the light irradiation unit 200, a digital data line at time intervals (A/D conversion intervals) as a reciprocal number of the A/D conversion rate since the light emission time. In this case, the light emission time may be determined with reference to the time when the optical pulse waveform becomes a peak.

The signal collection unit 140 is also referred to as a Data Acquisition System (DAS). According to the present specification, the concept of an electrical signal includes both an analog signal and a digital signal. 7 As described above, the signal collection unit 140 may be disposed inside the housing 181 of the probe 180. In such a configuration, information is transferred between the probe 180 and the computer 150 as digital signals, resulting in improved noise immunity. The use of high-speed digital signal transmission enables reducing the number of wiring and improving the operability of the probe 180 in comparison with analog signal transmission.

The addition averaging (described below) may also be performed by the signal collection unit 140. In this case, it is desirable to perform addition averaging by using hardware such as a FPGA.

### (Computer 150)

The computer 150 includes an acquisition unit 151, a storage unit 152, and a control unit 153. The acquisition unit 151 in charge of arithmetic/logic operations may include operational circuits including processors, such as a CPU and graphics processing unit (GPU), and a FPGA chip. These units may be configured by a single processor or operation circuit or a plurality of processors or operation circuits.

For example, the computer 150 sets a drive target waveform to control the drive unit 210 to drive a plurality of the serially-connected semiconductor light emission elements of the light irradiation unit 200 with at least two different optical pulse waveforms. More specifically, the computer 150 also serves as a light intensity setting unit for setting the light intensity of each of the at least two different optical pulse waveforms, and also serves as a light emission count setting unit for controlling the number of light emissions of the semiconductor light emission elements for emitting light having at least two different optical pulse waveforms.

In the following exemplary embodiment, the semiconductor light emission elements emit light with a fixed light intensity the same number of times. Alternatively, the exemplary embodiment can support light emission control other than the above-described control.

The computer 150 performs addition averaging of data at the same time since the light emission time of the digital data line output from the signal collection unit 140 in each light emission of the light irradiation unit 200. Then, the computer 150 stores the addition-averaged digital data line in the storage unit 152 as an addition-averaged electrical signal (combined photoacoustic signal) resulting from photoacoustic waves. Then, based on the combined photoacoustic signal stored in the storage unit 152, the acquisition unit 151 generates photoacoustic image data (structure images and function images) through image reconstruction and performs other various calculation processing. The acquisition unit 151 may receive various parameter inputs such as the sound speed of the subject 100 and the configuration of a holding unit from the input unit 170 and use them for calculations.

As a reconstruction algorithm used by the acquisition unit 151 to convert an electrical signal into three-dimensional volume data, arbitrary techniques such as a back-projection method in the time domain, a back-projection method in the Fourier domain, and a model base method (repetitive operation method) are applicable. Examples of back projection methods in the time domain include Universal Back-Projection (UBP), Filtered Back-Projection (FBP), and Delay-and-Sum.

The acquisition unit 151 may add a plurality of electrical signals acquired based on the acoustic waves received when a plurality of light irradiation units emit light a plurality of times and perform processing (addition averaging) for dividing the summation value by the number of light emissions of the light irradiation units.

The storage unit 152 includes nonvolatile memories such as a RAM and non-transitory storage media such as a ROM, magnetic disk, and flash memory. A storage medium storing programs is a non-transitory storage medium. The storage unit 152 includes a plurality of storage media.

The storage unit 152 can store various data including combined photoacoustic signals, photoacoustic image data generated by the acquisition unit 151, and reconstructed image data based on the photoacoustic image data.

The control unit 153 includes an operation element such as a CPU. The control unit 153 controls operations of each component of the photoacoustic apparatus 1. The control unit 153 transmits to the drive unit 210 a light emission control signal and an optical pulse waveform setting signal for a plurality of optical pulses. Then, the semiconductor light emission elements emit light having specified optical pulse waveforms to irradiate the subject 100 with the light. The control unit 153 also serves as an optical pulse waveform setting unit for setting optical pulse waveforms.

As described above, the control unit 153 controls optical pulse waveforms, the light intensity and the number of emissions of optical pulses, and the gain of photoacoustic signals based on a user instruction or in an automatic way. The control unit 153 also reads a program code stored in the storage unit 152 and controls operations of each component of the photoacoustic apparatus 1.

The control unit 153 also adjust the image on the display unit 160. In this configuration, oxygen saturation distribution images are sequentially displayed during the prove movement and photoacoustic measurement.

The computer 150 may be a workstation specially designed for the present exemplary embodiment. The computer 150 may also be a general-purpose personal computer (PC) or workstation which operates according to program instructions stored in the storage unit 152. Components of the computer 150 may be configured with different hardware components. At least a part of components of the computers 150 may be configured by a single hardware component.

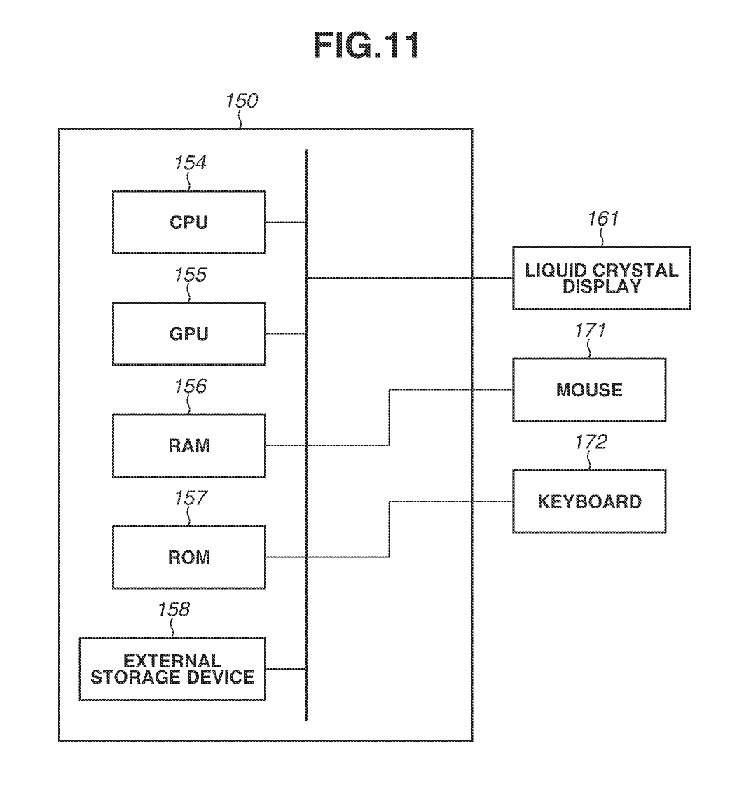

FIG. 11 illustrates an example of a specific configuration of the computer 150 according to the present exemplary embodiment. The computer 150 according to the present exemplary embodiment includes a CPU 154, a GPU 155, a RAM 156, a ROM 157, and an external storage device 158. The computer 150 is connected with a liquid crystal display (LCD) 161 as the display unit 160, and a mouse 171 and a keyboard 172 as the input unit 170.

The computer 150 and the reception unit 120 may be offered in a common housing. A computer provided in the housing may perform a part of signal processing, and a computer provided out of the housing may perform the remaining signal processing. In this case, the computers provided in and out of the housing can be collectively referred to as a computer according to the present exemplary embodiment. More specifically, hardware components of a computer do not need to be stored in one housing. The computer 150 may be implemented by an information processing apparatus which is offered in a cloud calculating service and installed at a remote site.

The computer 150 is equivalent to a processing unit of the present exemplary embodiment. In particular, the functions of the processing unit are implemented mainly by the acquisition unit 151.

### (Display Unit 160)

The display unit 160 is a liquid crystal display or an organic electro luminescence (EL) display. The display unit 160 is an apparatus for displaying images and numerical values of a specific position based on the subject information (for example, structure information and function information) acquired by the computer 150. The display unit 160 may display a GUI for operating an image and apparatus. The display unit 160 or the computer 150 may perform image processing (such as luminance value adjustment).

### (Input Unit 170)

The input unit 170 may be an operation console provided with a mouse and keyboard which can be operated by the user. The display unit 160 may be provided with a touch panel and used as the input unit 170. The input unit 170 receives instructions and numerical inputs from the user and transmits them to the computer 150.

Components of the photoacoustic apparatus 1 may be configured as separate apparatuses or as one integrated apparatus. At least a part of components of the photoacoustic apparatus 1 may be configured as one integrated apparatus.

The control unit 153 of the computer 150 also performs drive control of components included in the photoacoustic apparatus 1. The display unit 160 may display a GUI in addition to images generated by the computer 150. The input unit 170 is configured to allow the user to input information. Using the input unit 170, the user can perform operations such as an operation for starting/ending measurement, an operation for specifying an irradiation mode (described below), and an operation for storing a generated image.

### (Subject 100)

The subject 100 which is not a component of the photoacoustic apparatus 1 will be described below. The photoacoustic apparatus 1 according to the present exemplary embodiment can be used for the purpose of diagnosis and chemical treatment follow-up for malignant tumors and blood vessel diseases of human and animals. Therefore, the subject is assumed to be a diagnosis target portion of a living body, more specifically, breast, internal organs, blood vessel network, head, cervix, abdomen, fingers, and four limbs including fingers and toes. For example, if a human body is an object under measurement, then oxyhemoglobin, deoxyhemoglobin, a blood vessel or tumor containing a large amount of oxyhemoglobin or deoxyhemoglobin, or a new blood vessel formed near a tumor may be used as a target optical absorber. Plaque of a carotid wall may also be used as a target optical absorber. When the subject 100 is a human body, the melanin pigment contained in the skin may serve as an optical absorber which causes the above-described disturbance. Pigments such as methylene blue (MB) and indocyanine green (ICG), golden particulates, or a substance introduced from outside as an accumulation or chemical modification of these substances may be used as an optical absorber. A puncture needle or an optical absorber applied thereto may be used as an object under observation. The subject 100 may be an inanimate object such as a phantom and an object under test.

### (Operations of Exemplary Embodiment)

Then, the subject information acquisition apparatus according to the present exemplary embodiment irradiates the subject 100 with optical pulses having at least two different optical pulse waveforms a plurality of times, receives acoustic waves generated within the subject 100 in light irradiations, and acquires characteristic information about the inside of the subject based on the received photoacoustic signals. The first exemplary embodiment will be described in detail below centering on a configuration in which two sets of eight semiconductor light emission elements simultaneously emit two different optical pulse waveforms.

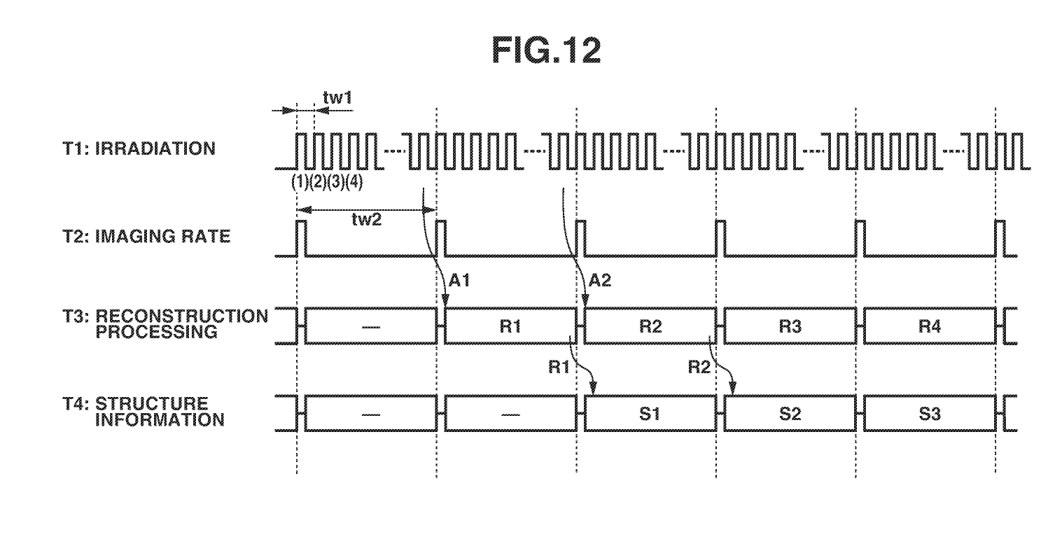

FIG. 12 is a timing diagram illustrating operations according to the first exemplary embodiment in an easy way. Referring to FIG. 12, the horizontal axis indicates time. These operations are controlled by the computer 150, FPGA, or dedicated hardware. A method used by the photoacoustic apparatus 1 according to the present exemplary embodiment to acquire a photoacoustic signal, and a method used by the photoacoustic apparatus 1 to generate a photoacoustic image based on the acquired photoacoustic signal will be described in detail below with reference to FIG. 12.

FIG. 12 is a timing diagram illustrating simultaneous emissions of optical pulses having two different optical pulse waveforms and the acquisition of structure information based on acquired photoacoustic signals. When acquiring blood vessel structure information, it is desirable to select about 795 nm as the wavelength of the light of the light irradiation unit 200 at which the absorption coefficients of oxyhemoglobin and deoxyhemoglobin are equal.

Since semiconductor emission elements have a low light intensity, the photoacoustic apparatus 1 performs the following control for the purpose of improving the S/N ratio. More specifically, the photoacoustic apparatus 1 controls a plurality of semiconductor light emission elements of the light irradiation unit 200 to repetitively emit light at irradiation intervals tw1, as illustrated by T1 in FIG. 12, and acquires photoacoustic signals. The length of the irradiation interval tw1 is set in consideration of Maximum Permissible Exposure (MPE) to the skin. For example, it is desirable to select 0.1 ms as the irradiation interval tw1.

As illustrated in FIG. 9, the light irradiation unit 200 includes 16 laser diodes as semiconductor light emission elements. Eight laser diodes (200a to 200h) and another eight laser diodes (200i to 200p) repetitively simultaneously emit light having the optical pulse waveforms illustrated in FIGS. 7A and 7C, respectively, at irradiation intervals tw1, and the acquisition unit 151 acquires photoacoustic signals. The total number and arrangements of laser diodes, and the number of laser diodes emitting light having two different optical pulse waveforms are to be considered as an example and may be configured in different ways. These factors may be determined by the specified light intensity and desired frequency characteristics.

Referring to T1 illustrated in FIG. 12, the laser diodes simultaneously emit optical pulses having the optical pulse waveforms illustrated in FIGS. 7A and 7C, and the acquisition unit 151 acquires photoacoustic signals 168 times ((1) to (4) . . . ) at irradiation intervals tw1, performs addition averaging of the photoacoustic signals, and acquires an addition-averaged photoacoustic signal A1 at imaging frame rate intervals tw2. More specifically, as described above, the laser diodes simultaneously emit light having the optical pulse waveforms illustrated in FIG. 7A and 7C to irradiate the subject 100 with optical pulses equivalent to the optical pulse waveforms illustrated in FIG. 7E. Then, the photoacoustic wave having the frequency spectrum illustrated in FIG. 7F can be obtained at imaging frame rate intervals tw2. Then, the acquisition unit 151 performs addition averaging of the 166 acquired photoacoustic signals to calculate the addition-averaged photoacoustic signal A1.

Referring to T3 illustrated in FIG. 12, the photoacoustic apparatus 1 performs the above-described reconstruction processing based on the addition-averaged photoacoustic signal A1 to obtain reconstructed image data R1. Then, the reconstructed image data R1 is sequentially output to the display unit 160 at imaging frame rate intervals tw2 (16.6 ms intervals) to be displayed as structure information S1 (refer to T4 illustrated in FIG. 12). The frequency of the imaging frame rate is about 60 Hz.

When the photoacoustic apparatus 1 acquires photoacoustic signals 166 times and performs addition averaging, the frequency of the imaging frame rate is about 60 Hz which coincides with the display frame rate of the display unit 160. If the number of addition averaging differs, the imaging frame rate may not coincide with the display frame rate. In this case, it is desirable to convert the imaging frame rate into a display frame rate by using a frame rate converter (not illustrated) and display data on the display unit 160.

As described above, it becomes possible to acquire information based on the frequency characteristics of a predetermined (desired) acoustic signal by simultaneously emitting light having at least two different optical pulse waveforms by using a part of a plurality of the semiconductor light emission elements of the light irradiation unit 200.

The result makes it possible to acquire information about the frequency characteristics of more suitable photoacoustic signals.

According to the first exemplary embodiment, the photoacoustic apparatus 1 was configured to perform control to simultaneously emit light having at least two different optical pulse waveforms. When performing addition averaging of photoacoustic signals acquired through a plurality of light emissions, the photoacoustic apparatus 1 may perform control to simultaneously emit light having at least two different optical pulse waveforms in a plurality of light emissions and to emit light having one optical pulse waveform in other light emissions, to irradiate the subject 100 with these optical pulses. Performing control in this way enables obtaining the frequency characteristics of a photoacoustic signal as a result of performing addition averaging of the frequency spectrum of a photoacoustic wave having one optical pulse waveform and the frequency spectrum of a photoacoustic wave obtained by simultaneously emit light having at least two different optical pulse waveforms, weighted by the number of light emissions.

### (Other Exemplary Embodiments)

According to the first exemplary embodiment, the photoacoustic apparatus 1 simultaneously emits light having at least two different optical pulse waveforms by using a part of a plurality of semiconductor light emission elements of the light irradiation unit 200 to irradiate the subject 100 with these optical pulses and acquires photoacoustic signals. As described above, it is also possible to change the number of semiconductor light emission elements corresponding to respective optical pulse waveforms out of a plurality of the semiconductor light emission elements. In addition, the photoacoustic apparatus 1 may be configured to simultaneously emit light while changing the light intensity of the semiconductor light emission elements for emitting optical pulses having respective optical pulse waveforms and acquire photoacoustic signals.

A plurality of wavelengths may be used as the wavelength of light emitted by the light irradiation unit 200. When a plurality of wavelengths is used, the oxygen saturation as function information can be calculated. According to the present exemplary embodiment, it is possible to, for each imaging frame rate, acquire photoacoustic signals by alternately switching between two different wavelengths, calculate reconstructed image data, and further calculate the oxygen saturation based on the reconstructed image data calculated for the two imaging frame rates. The calculation of the oxygen saturation is discussed in detail in Japanese Patent Application Laid-Open No. 2015-142740. When acquiring the structure information, the photoacoustic apparatus 1 simultaneously irradiates the subject 100 with light having at least two different optical pulse waveforms according to the present exemplary embodiment and obtains information about wide frequency characteristics. Then, optical pulse waveforms with other wavelengths for acquiring function information may have a shape of one optical pulse waveform with a long pulse width. This configuration makes it possible to clearly detect structure information regardless of the thickness of a blood vessel and acquire function information free from high-frequency components. Masking the function information with the structure information enables providing detailed information.

A plurality of the above-described exemplary embodiments may be implemented by one photoacoustic apparatus and selectively used. Further, the photoacoustic apparatus 1 according to the present exemplary embodiment may be additionally provided with a function of transmitting ultrasonic waves from transducers and performing measurement by using reflection waves. In this case, naturally, the light irradiation unit 200 does not emit light.

The photoacoustic apparatus according to another exemplary embodiment makes it possible to widen the frequency bandwidth of acoustic waves to be detected by irradiating the subject 100 with pulsed light at least a part of which have different pulse waveforms. This makes it possible to acquire information about substances ranging from a large-sized substance corresponding to a low-frequency to a small-sized substance corresponding to a high-frequency out of optical absorbers distributed over the subject 100. In addition, the frequency bandwidth of acoustic waves to be detected can be further widened by increasing the number of types of pulse waveforms.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Applications No. 2017-167255, filed Aug. 31, 2017, and No. 2017-167256, filed Aug. 31, 2017, which are hereby incorporated by reference herein in their entirety.

What is claimed is:

1. A photoacoustic apparatus comprising:
a light irradiation unit configured to irradiate a subject with pulsed light;
a reception unit configured to receive an acoustic wave generated when the subject is irradiated with the pulsed light and to convert the acoustic wave into an electrical signal;
a control unit configured to control the light irradiation unit to irradiate the subject with a plurality of optical pulses of the pulsed light; and
an acquisition unit configured to acquire information about the subject based on a plurality of the electrical signals converted from a plurality of acoustic waves generated when the subject is irradiated with the pulsed light a plurality of times,
wherein the control unit controls the light irradiation unit so that at least a part of the optical pulses to be emitted a plurality of times have a pulse waveform different from a pulse waveform of the other optical pulses.

2. The photoacoustic apparatus according to claim 1, wherein the control unit controls the light irradiation unit so that at least a part of the optical pulses to be emitted a plurality of times have a pulse width different from the other a pulse width of the other optical pulses.

3. The photoacoustic apparatus according to claim 1, wherein the control unit controls the light irradiation unit so that the number of light emissions is different between the optical pulses having different pulse waveforms.

4. The photoacoustic apparatus according to claim 1, wherein the control unit controls the light irradiation unit so that the light intensity is different between the optical pulses having different pulse waveforms.

5. The photoacoustic apparatus according to claim 1, wherein the acquisition unit performs processing of addition averaging of the plurality of electrical signals.

6. The photoacoustic apparatus according to claim 1, wherein the acquisition unit selects electrical signals having a predetermined frequency component from among a plurality of the electrical signals and performs addition averaging.

7. The photoacoustic apparatus according to claim 1, wherein the acquisition unit performs image reconstruction on each of the plurality of electrical signals to acquire a plurality of reconstructed images, and performs addition averaging of the plurality of reconstructed images.

8. The photoacoustic apparatus according to claim 1, wherein the acquisition unit performs image reconstruction on each of the plurality of electrical signals to acquire a plurality of reconstructed images, selects the reconstructed images corresponding to electrical signals having a predetermined frequency component from among the plurality of reconstructed images, and performs addition averaging.

9. The photoacoustic apparatus according to claim 1, wherein a peak frequency of a first acoustic wave of the plurality of acoustic waves and a peak frequency of a second acoustic wave of the plurality of acoustic waves are different from each other, the first acoustic wave being generated when the subject is irradiated with first pulsed light having a first pulse waveform among the pulsed light emitted a plurality of times, the second acoustic wave being generated when the subject is irradiated with second pulsed light having a second pulse waveform different from the first pulse waveform.

10. The photoacoustic apparatus according to claim 1, further comprising a filter unit configured to change the frequency characteristics of the electrical signals, based on a peak frequency of the acoustic wave.

11. The photoacoustic apparatus according to claim 10, wherein the filter unit has approximately the same characteristics as a frequency spectrum of the acoustic wave.

12. The photoacoustic apparatus according to claim 10, wherein the filter unit changes the frequency spectrum of the acoustic wave to approximately flat characteristics.

13. The photoacoustic apparatus according to claim 1, wherein the light irradiation unit includes a light source unit for generating the pulsed light.

14. The photoacoustic apparatus according to claim 13, wherein the light source unit includes a plurality of semiconductor light emission elements.

15. A photoacoustic apparatus comprising:
a plurality of light irradiation units configured to irradiate a subject with pulsed light;
a control unit configured to control the plurality of light irradiation units to irradiate the subject with the pulsed light from the plurality of light irradiation units at approximately the same time;
a reception unit configured to receive an acoustic wave generated when the subject is irradiated with the pulsed light and convert the acoustic wave into an electrical signal; and
an acquisition unit configured to acquire information about the subject based on the electrical signal,
wherein the control unit controls the plurality of light irradiation units to provide the optical pulses emitted from the plurality of light irradiation units with pulse waveforms different from each other.

16. The photoacoustic apparatus according to claim 15, wherein the control unit controls the plurality of light irradiation units so that the optical pulses to be emitted from the plurality of light irradiation units have pulse widths different from each other.

17. The photoacoustic apparatus according to claim 16, wherein the pulse width is 10 ns or more and 1 μs or less.

18. The photoacoustic apparatus according to claim 17, wherein the pulse width is 100 ns or more and 800 ns or less.

19. The photoacoustic apparatus according to claim 15, wherein the control unit controls the plurality of light irradiation units so that the optical pulses to be emitted from the plurality of light irradiation units have peak intensities different from each other.

20. The photoacoustic apparatus according to claim 15, wherein the light irradiation units include a light source unit for generating the pulsed light.

21. The photoacoustic apparatus according to claim 20, wherein the light source unit includes a plurality of semiconductor light emission elements.

22. The photoacoustic apparatus according to claim 21, wherein the control unit controls a luminescence intensity of the plurality of semiconductor light emission elements to control the peak intensity of the pulsed light.

23. The photoacoustic apparatus according to claim 21, wherein the control unit controls the number of light emission elements to be driven to emit light among the plurality of semiconductor light emission elements to control the peak intensity of the pulsed light.

24. The photoacoustic apparatus according to claim 15, wherein the acquisition unit performs processing for adding a plurality of electrical signals acquired based on the acoustic waves received when the plurality of light irradiation units emits light a plurality of times and dividing a summation value by the number of light emissions of the light irradiation units.
